(19) United States
(12) Patent Application Publication
(10) Pub. No.: US 2005/0282985 A1
(43) Pub. Date: Dec. 22, 2005

(54) FLUORINE-ATOM-CONTAINING POLYMERIZABLE UNSATURATED-MONOMER, FLUORINE-ATOM-CONTAINING POLYMERIC COMPOUND AND PHOTORESIST RESIN COMPOSITION

(76) Inventors: Hiroshi Koyama, Himeji-shi (JP); Kiyoharu Tsutsumi, Himeji-shi (JP)

Correspondence Address: BIRCH STEWART KOLASCH & BIRCH, PO BOX 747, FALLS CHURCH, VA, 22040-0747, US

(21) Appl. No.: 10/528,111

(22) PCT Filed: Jun. 17, 2004
(86) PCT No.: PCT/JP04/08846

§ 371 Date: Mar. 16, 2005

(51) Int. Cl. C08F 116/12; C07C 69/52

(52) U.S. Cl. 526/247; 526/266; 526/282; 560/220

(57) ABSTRACT

A fluorine-atom-containing polymerizable unsaturated-monomer of the present invention is represented by the following formula (1a) or (1b): 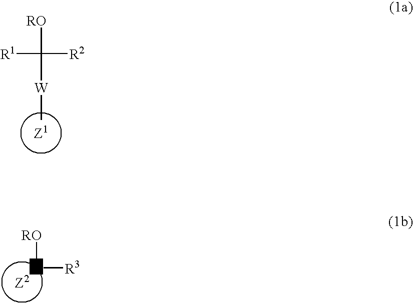
wherein each of a ring Z¹ and a ring Z² is an alicyclic carbon ring; when the alicyclic carbon ring is multi-cyclic, a part of atoms constituting the ring may be substituted by an oxygen atom, a sulfur atom or a nitrogen atom; an atom constituting the ring Z¹ and the ring Z² may have a substituent; each of R¹, R² and R³ is an alkyl group or a fluoroalkyl group; R is an specific unsaturated acyl group; provided that at least one of R¹ and R² is a fluorine atom or a fluoroalkyl group in the formula (1a), and in the formula (1b) (i) the ring Z² is bonded by a fluorine atom or a fluoroalkyl group, or (ii) R³ is a fluoroalkyl group.

### TECHNICAL FIELD

The present invention relates to an useful polymerizable unsaturated monomer containing a fluorine atom and a production process thereof for as a monomer component of a photoresist resin used for a micro processing of semiconductor and others, a polymeric compound containing a repeated unit corresponding to the monomer, a photoresist resin composition containing the polymeric compound and a process of producing a semiconductor using the resin composition.

### BACKGROUND ART

An exposure-light source of lithography used in semiconductor manufacture becomes shorter wavelength year after year and is converted from KrF excimer laser having a wavelength of 247 nm to ArF excimer laser having a wavelength of 193 nm. F₂ excimer laser having a wavelength of 157 nm is hopefully focused as an exposure-light source of next generation. A conventional resin used in a resist for KrF excimer laser exposure and ArF excimer laser exposure doesn't show sufficient permeability against a vacuum ultraviolet light (a light having a wavelength of 190 nm or low). Some polymeric compounds having a fluorine atom in the molecule has been proposed as a resin with a high transparency against such a vacuum ultraviolet light (for example, Japanese Unexamined Patent Application Publication No. 2002-6501, Japanese Unexamined Patent Application Publication No. 2002-155118, Japanese Unexamined Patent Application Publication No. 2002-179731, Japanese Unexamined Patent Application Publication No. 2002-220419, Japanese Unexamined Patent Application Publication No. 2002-293840, Japanese Unexamined Patent Application Publication No. 2002-327013, Japanese Unexamined Patent Application Publication No. 2003-2925 and so on). However even those resins are not necessarily sufficient for transparency (transparency) against a vacuum ultraviolet light.

Further, resins having appropriate such as, in addition to transparency against a light used to exposure, a function allowing exposed part in light exposure to change to alkali-soluble by an acid (acid-elimination function), resistance to dry etching (etching resistance) and adhesion to substrate (substrate adhesion) in balance rarely exists.

### DISCLOSURE OF INVENTION

An object of the present invention is to provide a useful and novel fluorine-atom-containing polymerizable unsaturated-monomer and a production process of thereof to obtain a polymeric compound having high transparency against a light having a wavelength of 300 nm or less, particularly a vacuum ultraviolet light such as F2 exima laser (157 nm).

Another object of the present invention is to provide a novel fluorine-atom-containing polymerizable unsaturated-monomer and a production process of thereof which can give high transparency, superior acid-eliminating function and etching resistance and, further can be easily co-polymerize another co-monomer to give various functions required as a photoresist.

Further, another object of the present invention is to provide a polymeric compound having high transparency against a light having a wavelength of 300 nm or less, particularly a vacuum ultraviolet light, a photoresist resin composition containing the polymeric compound and a process for producing a semi-conductor using the resin composition.

Another object of the present invention is to provide a polymeric compound having high transparency against a light used in exposure and further having various functions such as acid-eliminating function, etching resistance and substrate adhesion in balance, a photoresist resin composition containing the polymeric compound and a process for producing a semi-conductor using the resin composition.

In addition, another object of the present invention is to provide a photoresist resin composition which can be allowed to form a micro pattern in high accuracy and a process for producing a semi-conductor using the resin composition. The present inventors made intensive investigations to find that a novel fluorine-atom-containing unsaturated carboxylic acid ester containing a acid-eliminating function group having a specific alicyclic hydrocarbon ring at a portion of alcohol and further this compound can be easily co-polymerized with another monomer which can give various functions required as a photoresist and can be obtained a polymeric compound by this co-polymerization which is a high transparency against a light having various functions such as a wavelength of 300 nm or less, particularly a vacuum and further acid-eliminating function, etching resistance and substrate adhesion in balance. The present invention was achieved based on these discoveries.

Namely, the present invention provides a fluorine-atom-containing polymerizable unsaturated-monomer represented by the following formula (1a) or (1b): 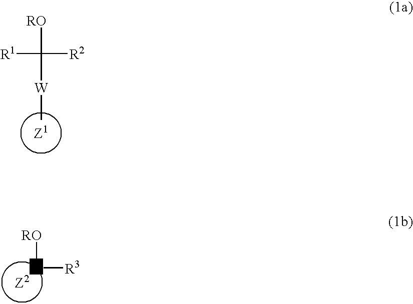
wherein each of a ring Z¹ and a ring Z² is an alicyclic carbon ring; when the alicyclic carbon ring is multi-cyclic, a part of atoms constituting the ring may be substituted by an oxygen atom, a sulfur atom or a nitrogen atom; an atom constituting the ring Z¹ and the ring Z² may have a substituent; each of R¹, R² and R³ is an alkyl group or a fluoroalkyl group; W denotes a single bond or a combining group and R is an unsaturated acyl group represented by the following formula (2): 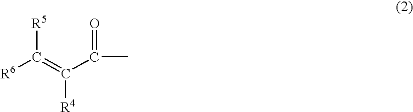
wherein each of R⁴, R⁵ and R⁶ is a hydrogen atom, a fluorine atom, an alkyl group or a fluoroalkyl group; a carbon atom bonded by at least one hydrogen atom exists in an adjacent position to the carbon atom bonded by an RO— group represented by the formula (1a) and the formula (1b); provided that at least one of R¹, R², R⁴, R⁵ and R⁶ is a fluorine atom or a fluoroalkyl group in the formula (1a), and in the formula (1b) (i) the ring Z² is bonded by a fluorine atom or a fluoroalkyl group, or (ii) R³ is a fluoroalkyl group.

In the above fluorine-atom-containing polymerizable unsaturated-monomers, a compound in which neither of adjacent carbon atoms to the carbon atom bonded by an RO— group has a fluorine atom in the formula (1a) or the formula (1b) is preferable.

In the formula (1a), it is preferable that (i) the ring Z¹ is bonded by a fluorine atom or a fluoroalkyl group; (ii) at least one of R and R² is a fluoroalkyl group having a hydrogen atom at 1st position; (iii) both of R¹ and R² are alkyl groups which have a hydrogen atom at 1st position and may be fluorinated; or (iv) at least one of R¹ and R² is an alkyl group of which a carbon number is three or more and may be fluorinated.

The ring Z¹ or the ring Z² include a ring represented by the following formula (3a), (3b) or (3c); 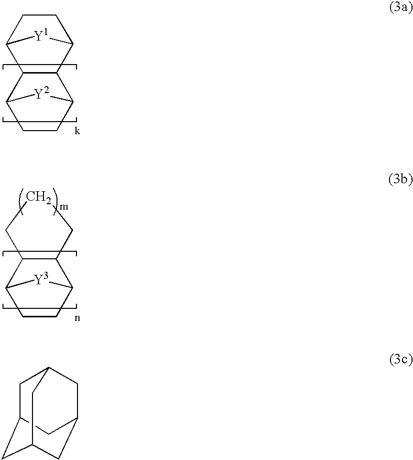
wherein Y¹ is an alkylene group, an oxygen atom or a sulfur atom, each of Y² and Y³ is an alkylene group, an oxygen atom, a sulfur atom or non bonding, each of k and n denotes an integer of 0 to 3, m denotes 1 or 2, and an atom constituting the rings in the formulae may have a substituent.

The present invention, further, provides a process for producing a fluorine-atom-containing polymerizable unsaturated-monomer wherein a compound represented by the following formula (1a): 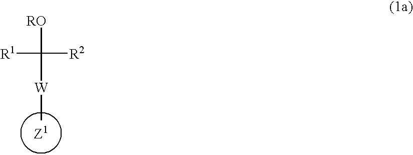
wherein each of the ring Z¹ is an alicyclic carbon ring, R¹ is an alkyl group or a fluoroalkyl group, R² is an alkyl group or a fluoroalkyl group, R is an unsaturated acyl group represented by the following formula (2): 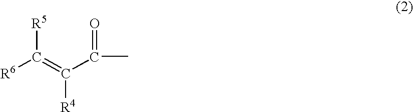
wherein each of R⁴, R⁵ and R⁶ is a hydrogen atom, a fluorine atom, an alkyl group or a fluoroalkyl group, and W denotes a single bond or a combining group; provided that, when a compound represented by the formula (4b) is used as a raw material, R¹═R²; and a carbon atom bonded by at least one hydrogen atom exists in an adjacent position to the carbon atom bonded by an RO— group;
is obtained by allowing a compound represented by the following formula (4a) or (4b) 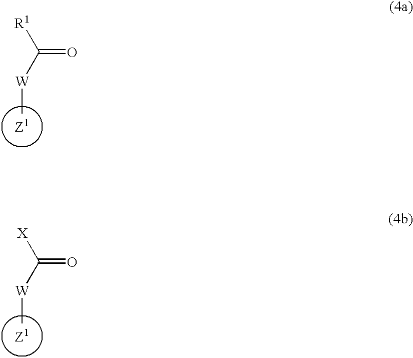
   wherein a ring Z¹ is the same as above; when the alicyclic carbon ring is multi-cyclic, a part of atoms constituting the ring ay be substituted by an oxygen atom, a sulfur atom or a nitrogen atom; an atom constituting the ring Z¹ may have a substituent;
R¹ is the same as above; X is a halogen atom; W is the same as above;
to react with a (fluoro) alkylating agent represented by the following formula (5):
   R²-M (5)
   wherein R² is the same as above; M is a metal atom or a —MgX¹ group, wherein X¹ is a halogen atom; provided that a carbon atom bonded by at least one hydrogen atom exists in a adjacent position to a carbonyl group in the above formula (4a) or (4b), or in 1st position of R² in the formula (5);
and allowing an acyl halide represented by the following formula (6) to react to the obtained reaction product:
   R—X² (6)
   wherein R is the same as above; and X² is a halogen atom; provided that at least one of R¹, R², R⁴, R⁵ and R⁶ is a fluorine atom or a fluoroalkyl group.

In addition, the present invention provides a process for producing a fluorine-atom-containing polymerizable unsaturated-monomer wherein a compound represented by the following formula (1b): 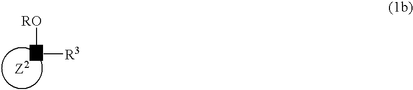
wherein the ring Z² is an alicyclic carbon ring, R³ is an alkyl group or a fluoroalkyl group, and R is an unsaturated acyl group represented by the following formula (2): 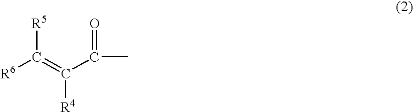
wherein each of R⁴, R⁵ and R⁶ is a hydrogen atom, a fluorine atom, an alkyl group or a fluoroalkyl group; and a carbon atom bonded by at least one hydrogen atom exists in an adjacent position to the carbon atom bonded by an RO— group;
is obtained by allowing a carbonyl compound represented by the following formula (7): 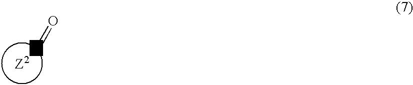
   wherein a ring Z² is the same as above; when the alicyclic carbon ring is multi-cyclic, a part of atoms constituting the ring may be substituted by an oxygen atom, a sulfur atom or a nitrogen atom; an atom constituting the ring Z² may have a substituent; to react with a (fluoro) alkylating agent represented by the following
   Formula (8):
   R³-M (8)
   wherein R³ is the same as above; M is a metal atom or a —MgX¹ group, wherein X¹ is a halogen atom; provided that a carbon atom bonded by at least one hydrogen atom exists in a adjacent position to a carbonyl group in the above formula (7) or in 1st position of R³ in the formula (8);
and allowing an acyl halide represented by the following formula (6) to react to the obtained reaction product:
   R—X² (6)
   wherein R is the same as above; and X² is a halogen atom.

Further, the present invention provides a polymeric compound having a repeated unit corresponding to the above fluorine-atom-containing polymerizable unsaturated-monomer.

The above polymeric compound, in addition, may contain a repeated unit having a substrate-adhesive function and/or a hydrophilic function.

Further, the above polymeric compound may contain a repeated unit corresponding to a vinyl ether monomer represented by the following formula (9a) or (9b): 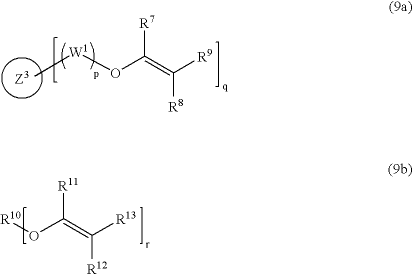
wherein, in the formula (9a), the ring Z³ is one of rings represented by the following formula (10a), (10b), (10c), (10d), (10e), (10f), (10g), (10h) or (10i): 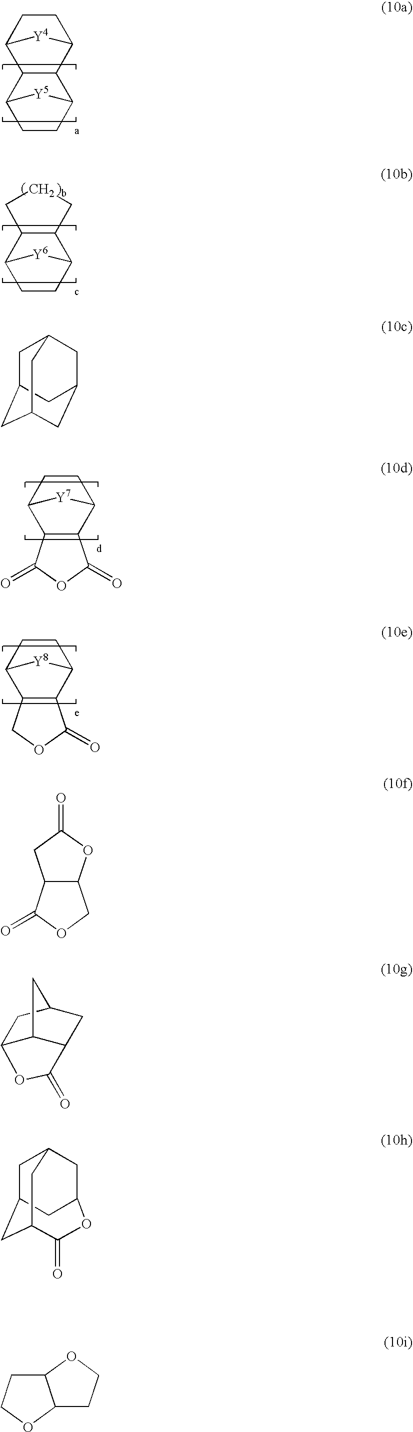
wherein Y⁴ is an alkylene group, an oxygen atom or a sulfur atom; each of Y⁵, Y⁶, Y⁷ and Y⁸ is an alkylene group, an oxygen atom, sulfur atom or non-bonding; each of a, c, d and e denotes an integer of 0 to 3; b denotes 1 or 2; and rings of the formulae may have a substituent; W¹ is a bivalent hydrocarbon group; each of R⁷, R⁸ and R⁹ is identical to or different from a hydrogen atom or an organic group; at least two of a ring Z³, W¹, R⁷, R⁸ and R⁹ may be combined together to constitute a ring with one or two or more adjacent atoms; p denotes 0 or 1 and q denoted an integer 1 to 8; when q is 2 or more, each of groups in q pairs of brackets may be identical to or different from; in the formula (9b), R¹⁰ is an alkyl group which may have a substituent and each of R¹¹, R¹² and R¹³ is identical to or different from a hydrogen atom or an organic group; at least two of R¹⁰, R¹¹, R¹² and R¹³ may be combined together to constitute a ring with one or two or more adjacent atoms; r denotes an integer of 1 to 8; when r is 2 or more, each of groups in r pairs of brackets may be identical to or different from.

The above polymeric compound may contain a repeated unit corresponding to an acryl monomer represented by the following formula (11a), (11b), (11c) or (11d): 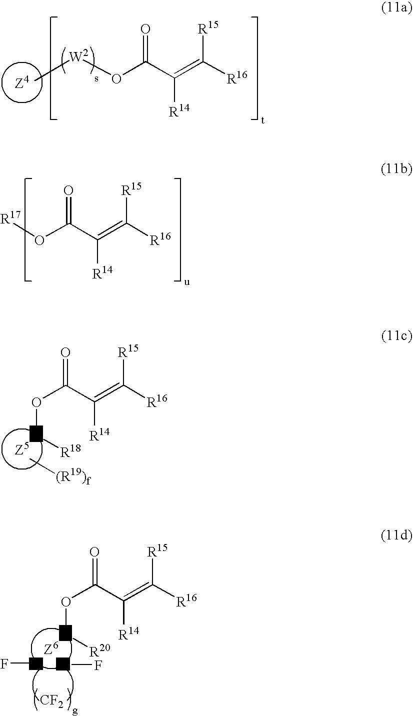
wherein, in the formula (11a), the ring Z⁴ is one of rings presented by the following formula (10a), (10b), (10c), (10d), 10e), (10f), (10g), (10h) or (10i): 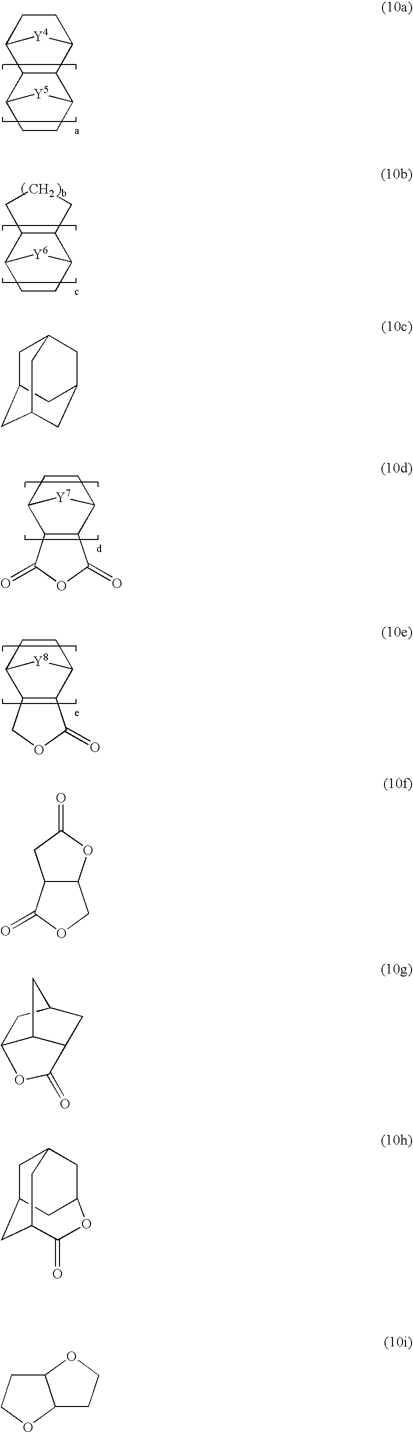
wherein Y⁴ is an alkylene group, an oxygen atom or a sulfur atom; each of Y⁵, Y⁶, Y⁷ and Y⁸ is an alkylene group, an oxygen atom, sulfur atom or non-bonding; each of a, c, d and e denotes an integer of 0 to 3; b denotes 1 or 2; and rings of the formulae may have a substituent; W² is a bivalent hydrocarbon group; provided that each of Z⁴ and W² does not have a fluorine atom or a fluoroalkyl group as a substituent; each of R¹⁴, R¹⁵ and R¹⁶ is a hydrogen atom, a fluorine atom, an alkyl group or a fluoroalkyl group; s denotes 0 or 1 and t denotes an integer of 1 to 8; when t is 2 or more, each of groups in t pairs of brackets may be identical to or different from; in the formula (11b), R¹⁷ is an alkyl group which may have a substituent and each of R¹⁴, R¹⁵ and R¹⁶ is the same as above; u denotes an integer of 1 to 8; when u is 2 or more, each of groups in u pairs of brackets may be identical to or different from; in the formula (11c), a ring Z⁵ is an alicyclic carbon ring which may have a substituent; R¹⁸ is a hydrogen atom, a fluorine atom, an alkyl group or a fluoroalkyl group and R¹⁹ is a fluoroalkyl group; each of R¹⁴, R¹⁵ and R¹⁶ is the same as above; f denotes an integer of 1 to 6; in the formula (11d), a ring Z⁶ is an alicyclic carbon ring which may have a substituent; R²⁰ is a hydrogen atom, a fluorine atom, an alkyl group or a fluoroalkyl group; each of R¹⁴, R¹⁵ and R¹⁶ is the same as above; g denotes 3 or 4; provided that compounds represented by the formulae (1a) and (1b) are excepted for.

The above polymeric compound a repeated unit corresponding to a cyclic unsaturated monomer represented by the following formula (12a) or (12b); 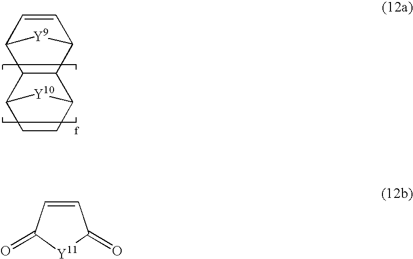
wherein each of Y9 and Y10 is an alkylene group, an oxygen atom, a sulfur atom or a non-bonding; Y11 is an oxygen atom or a —NH— group; f denotes an integer of 0 to 3; an atom constituting a ring of the formulae may have a substituent.

The present invention, further, provides a photoresist resin composition, comprising at least the above polymeric compound and a photo acid generator.

In addition, the present invention provides a process of producing a semiconductor, comprising the steps of applying the above photoresist resin composition onto a base or substrate to form a resist film, exposing, developing and thereby produce a pattern.

Further, in a vinyl ether monomer in the present description, a compound in which a hydrogen atom in the vinyl group is substituted by a substituent is also included. In addition, a α,β-unsaturated carboxylic acid ester monomer may be described as an acrylic acid ester monomer or an acryl monomer for the sake of convenience. Further, in the present description, the term “organic group” is used as, not only a group having a carbon atom, a broad meaning containing a group having a non-metal atom such as, for example, a halogen atom, a nitro group and a sulfonic acid group.

According to the present invention, a useful and novel fluorine-atom-containing polymerizable unsaturated-monomer and a production process of thereof to obtain a polymeric compound having high transparency against a light having a wavelength of 300 nm or less, particularly a vacuum ultraviolet light such as F2 excimer laser (157 nm) is provide. In addition, a novel fluorine-atom-containing polymerizable unsaturated-monomer and a production process of thereof which can give high transparency, superior acid-eliminating function and etching resistance and, further can be easily co-polymerize another co-monomer to give various functions required as a photoresist is provided.

A polymeric compound of the present invention has high transparency against a light having a wavelength of 300 nm or less, particularly a vacuum ultraviolet light. Further, in case of using to a photoresist, various functions such as acid-eliminating function, etching resistance and substrate adhesion are performed in balance. Therefore, by a photoresist resin composition containing the said polymeric compound and a process of producing a semi-conductor using the said photoresist resin composition, a micro pattern can be formed high accurately.

### BEST MODE FOR CARRYING OUT THE INVENTION

[Fluorine-Atom-Containing Polymerizable Unsaturated-Monomer]

A fluorine-atom-containing polymerizable unsaturated-monomer of the present invention is an acrylic acid ester compound represented by the above formula (1a) or (1b). This monomer provides a polymeric compound by polymerizing at an unsaturated position of an unsaturated acyl group (R—). Because this monomer has a fluorine atom in its molecule, it provides high transparency to a polymer against light of wavelength of 300 nm or lower, particularly vacuum ultraviolet light, and further because it has an alicyclic hydrocarbon ring in its molecule, it can provide superior etching resistance. Further, because this monomer has a carbon atom bonded by at least one hydrogen atom at an adjacent position to a carbon atom bonded by an unsaturated acyloxy group (RO— group), an elimination reaction caused by an acid proceeds to generate a carboxylic acid and an olefin compound corresponding to the said unsaturated acyloxy group and become to alkali-soluble and therefore an acid-elimination-providing property is had. In addition, the monomer of the present invention is easy to copolymerize with various monomers used for providing various functions (for example, substrate adhesion, transparency, etching resistance and others) demanded as a photoresist, for example, other acryl monomers, a vinyl ether monomer, a cyclic unsaturated monomer and others. Therefore, a polymeric compound having, for example, superior transparency against vacuum ultraviolet light and further having well-balanced properties such as acid-elimination, substrate adhesion and etching resistance can be prepared easily.

In the formula (1a), a ring Z¹ is an alicyclic hydrocarbon ring. The alicyclic hydrocarbon ring isn't particularly limited and may be both of a single ring (a 3 to 20 membered single ring and others) and a multiple ring (a bridged ring) (a multiple ring having 2 to 5 rings and others). Provided that, when the alicyclic hydrocarbon ring is a multiple ring, a part of atoms (for example, about 1 to 3 carbon atoms) constituting the ring may be substituted by an oxygen atom, a sulfur atom or a nitrogen atom. In this case, a ring bonded by a group having an unsaturated acyl group represented by the formula (2) is preferable to be constituted by a hydrocarbon ring.

As typical examples of a ring Z¹, there may be mentioned rings represented by the above formula (3a), (3b) or (3c). In the formulae, Y¹ is an alkylene group, an oxygen atom or a sulfur atom and each of Y² and Y³ is an alkylene group, an oxygen atom, a sulfur atom or non-bonding. As the alkylene group, there may be mentioned, for example, an alkylene group of a linear or branched chain of 1 to 3 carbon atoms (preferably 1 to 2) such as a methylene, an ethylene, a dimethylmethylene, a propylene and a trimethylene group. As Y¹ and Y², an alkylene group of 1 to 3 carbon atoms such as a methylene group or an oxygen atom is particularly preferable. As Y³, an alkylene group of 1 to 3 carbon atoms such as a methylene group, an oxygen atom or non-bonding is particularly preferable. Each of k and n denotes an integer of 0 to 3, preferably 0 to 2, and more preferably 0 or 1. m denotes 1 or 2. Atoms constituting the rings of the formulae (3a) to (3c) may have a substituent.

A ring represented by the formula (3a) (the insides of following parentheses mean a cyclic group name) includes, for example, a norbornane ring (such as a norbornane-2-yl group), a tetracyclo[4.4.0.1^{2,5}.1^{7,10}]dodecane ring (such as a tetracyclo[4.4.0.1^{2,5}.1^{7,10}]dodecane-3-yl group), a 7-oxabicyclo[2.2.1]heptane ring (such as 7-oxabicyclo[2.2.1]heptane-2-yl group) and others.

The ring represented by the formula (3a) (the insides of following parentheses mean a cyclic group name) includes, for example, a cyclopentane ring (a cyclopentyl group), a cyclohexane ring (a cyclohexyl group), a perhydroindene ring (a perhydroindene-1-yl group), a decalin ring (such as a decaline-1-ly group), a perhydrofuluorene ring (such as a perhydrofuluorene-1-yl group), a tricyclo[5.2.1.0^{2,6}]decane ring (such as a tricyclo[5.2.1.0^{2,6}]decane-3-yl group), a tricyclo[6.2.1.0^{2,7}]undecane ring (such as a tricyclo[6.2.1.0^{2,7}]undecane-3-yl group) and others.

The ring represented by the formula (3c) (the insides of following parentheses mean a cyclic group name) includes an adamantane ring (such as a adamantine-1-yl group and a adamantine-3-yl group).

An atom constituting a ring Z¹ may have a substituent. As the substituent, there may be mentioned, for example, a halogen atom, an alkyl group, a haloalkyl group, an aryl group, a hydroxyl group which may be protected by a protecting group, a hydroxy(halo)alkyl group which may be protected by a protecting group, an amino group which may be protected by a protecting group, a carboxyl group which may be protected by a protecting group, a sulfo group which may be protected by a protecting group, an oxo group, a nitro group, a cyano group, an acyl group which may be protected by a protecting group and others.

As the said halogen atom, there may be mentioned, for example, a fluorine, a chlorine, a bromine atom and others.

As the alkyl group, there may be mentioned, for example, a alkyl group of C₁ to C₁₀ (preferably a alkyl group of C₁ to C₅) such as a methyl, an ethyl, a propyl, a butyl, an isobutyl, a s-butyl, a t-butyl, a hexyl, an octyl and a decyl group and others. As the haloalkyl group, there may be mentioned, for example, a chloroalkyl group such as a chloromethyl group; a fluoroalkyl group (e.g., a fluoroalkyl group of C₁ to C₁₀, preferably a C₁₋₅ fluoroalkyl group) such as a trifluoromethyl, a 2,2,2-trifluoroethyl and a pentafluroethyl group; and others. As the aryl group, there may be mentioned, for example, a phenyl, a naphtyl and others. An aromatic ring of aryl group may have a substituent such as a halogen atom such as a fluorine atom, an alkyl group of C₁ to C₄ such as a methyl group, a haloalkyl group of C₁ to C₅ of such as a trifluoromethyl group, a hydroxyl group, an alkoxy group of C₁ to C₄ such as a methoxy group, an amino group, a dialkylamino group, a carboxyl group, an alkoxycarbonyl group such as a methoxycarbonyl group, a nitro group, a cyano group, an acyl group such as an acetyl group. As the hydroxyalkyl group, there may be mentioned, for example, a hydroxymethyl, a hydroxymethyl, a hydroxypropyl, a 1-hydroxy-1-methylethyl group, a 2,2,2-trifluoro-1-trifluoromethyl-1-hydroxyethyl group and others [preferably a hydroxy alkyl group of C₁ to C₄, hydroxyhaloalkyl group of C₁ to C₄ and others.

As a protecting group of the said hydroxyl group and a hydroxyl group in the hydroxy(halo)alkyl group, a common protecting group in the field of organic synthesis, for example, an alkenyl group (e.g., an alkyl group of C₁ to C₄ such as a methyl and a t-butyl group and others), an alkenyl group (e.g., an allyl group and others), a cycloalkyl group (e.g., a cyclohexyl group and others), an aryl group (e.g., 2,4-dinitrophenyl group and others), an aralkyl group (e.g., a benzyl group and others); a group which can form an acetal or hemi-acetal group with a hydroxyl group such as a substituted methyl group (e.g., a methoxymethyl, a methylthiomethyl, a benzyloxymethyl, a t-butoxymethyl, a 2-methoxyethoxymethyl group and others), a substituted ethyl group (e.g., a 1-ethoxyethyl and others), a tetrahydropyranyl group, a tetrahydrofuranyl group, a 1-hydroxyalkyl group (e.g., a 1-hydroxyethyl group and others); an acyl group (e.g., a aliphatic acyl group of C₁ to C₆ such as a formyl, an acetyl, a propyl, a butyryl, an isobutyryl, a pivaloyl group; an acetoacetyl group; an aromatic acyl group such as a bezoyl group; and others), an alkoxycarbonyl group (e.g., an alkoxy-carbonyl group of C₁ to C₄ such as a methoxycarbonyl group and others), an aralkyloxycarbonyl group, a substituted or non-substituted carbamoyl group, a substituted silyl group (e.g., a trimethylsilyl group and others), and a bivalent hydrocarbon group (e.g., a methylene, an ethylidene, an isopropylidene, a cyclopentylidene, a cyclohexylidene, a benzylidene group and others) which may have a substituent when there are two or more hydroxyl groups (including a hydroxymethyl group) in the molecule can be exemplified.

As the said protecting group of amino group, there may be mentioned, for example, an alkyl group, an aralkyl group, an acyl group, an alkoxycarbonyl group and others which are exemplified as the said protecting group of hydroxyl group. Further, the protecting group of carboxyl group and sulfo group, there may be mentioned, for example, an alkoxy group (e.g., an alkoxy group of C₁ to C₆ such as a methoxy, a ethoxy and a butoxy group), a cycloalkyloxy group, an aryloxy group, an aralkyloxy group, a trialkylsilyloxy group, an amino group which may have a substituent, a hydrazino group, an alkoxycarbonylhydrazino group, an aralkylcarbonylhydrazino group and others.

As the said acyl group, there may be mentioned, for example, a aliphatic acyl group of C₁ to C₆ such as a formyl, an acetyl, a propionyl, a butyryl, an isobutyryl and a pivaloyl group; an acetoacetyl group; an aromatic acyl group such as a benzoyl group; and others. As a protecting group of acyl group, a common protecting group of acyl group in the field of organic synthesis can be used. As a protected pattern of acyl group, there may be mentioned, for example, an acetal (including a hemiacetal) and others.

When a number of a substituent of the ring Z¹ is two or more, they bond each other and may form a 4-membered ring, for example a cycloalkane ring, a lactone ring and others, with a carbon atom constituting the ring Z¹. These rings may have a substituent (the same substituent as a substituent which may be had by an atom constituting the said ring Z¹) which is a halogen atom such as a fluorine atom and others.

In the above substituents, a fluorine atom, a fluoroalkyl group (e.g., a fluoroalkyl group of C₁ to C₁₀ such as a trifluoromethyl, a 2,2,2-trifluoroethyl and a pentafluoroethyl group, particularly, a fluoroalkyl group of C₁ to C₅ and others), an alkyl group (e.g., a alkyl group of C₁ of C₁₀ such as a methyl, a ethyl, a propyl, an isopropyl and a butyl group and others) and others are preferable. A number of substituents in the ring Z¹ is about 0 to 5, preferably, about 0 to 3.

In the formula (1a), each of R¹ and R² is an alkyl group or a fluoroalkyl group. In the present description, the meaning summing an alkyl group and a fluoroalkyl group may be called as “an alkyl group which may be fluorinated”. Each of R¹ and R² may be a same group or a different group each other. As the said alkyl group, there may be mentioned, for example, a linear- or branched-chain alkyl group of about 1 to 15 carbon atoms (preferably, about 1 to 12) such as a methyl, an ethyl, a propyl, an isopropyl, a butyl, an isobutyl, a s-butyl, a t-butyl, a pentyl, an isopentyl, a hexyl, a heptyl, an octyl, a nonyl, a decyl, and a dodecyl group and others.

As the said fluoroalkyl group, there may be mentioned, for example, a linear- or branched-chain fluoroalkyl group of about 1 to 15 carbon atoms (preferably, about 1 to 12 and more preferably, about 2 to 10) which is substituted by a fluorine atom to at least one hydrogen atom of the said alkyl group and others. As typical examples of such fluoroalkyl groups, a trifluoromethyl, a pentafluoroethyl, a 2,2,2-trifluoroethyl, 2,2,2-trifluoro-1-(trifluoromethyl)ethyl, a heptafluoropropyl, 2,2,3,3,3-pentafluoropropyl, a 2,2,3,3-tetrafluoropropyl, a nonafluorobutyl, a 2,2,3,3,4,4,4-heptafluorobutyl, a 2,2,3,3,4,4-hexafluorobutyl, an undecafluoropentyl, a 2,2,3,3,4,4,5,5,5-nonafluoropentyl, a 2,2,3,3,4,4,5,5-octafluoropentyl, a tridecafluorohexyl, a 2,2,3,3,4,4,5,5,6,6,6-undecafluorohexyl, a 2,2,3,3,4,4,5,5,6,6-decafluorohexayl, a 3,3,4,4,5,5,6,6,6-nonafluorohexyl, a pentadecafluoroheptyl, a 2,2,3,3,4,4,5,5,6,6,7,7,7-tridecafluoroheptyl, a 2,2,3,3,4,4,5,5,6,6,7,7-dodecafluoroheptyl, a heptadecafluorooctyl, a 2,2,3,3,4,4,5,5,6,6,7,7,8,8,8-pentadecafluorooctyl, a nonadecafluorononyl, a 2,2,3,3,4,4,5,5,6,6,7,7,8,8,9,9,9-heptadecafluorononyl, a heneicosafluorodecyl, a 2,2,3,3,4,4,5,5,6,6,7,7,8,8,9,9,10,10,10-nonadecafluorodecyl group and others.

In the formula (1a), W is a single bond or a combining-group. As the combining group, there may be mentioned, for example, a bivalent hydrocarbon group which may have a substituent, an ether bond (an oxygen atom), a thioether (a sulfur atom), a carbonyl group, a thiocarbonyl group, a —NH— group which may be substituted, a bivalent group which is bonded by plural numbers of these groups and others.

The said bivalent hydrocarbon group includes a bivalent aliphatic hydrocarbon group, a bivalent alicyclic hydrocarbon group, a bivalent aromatic hydrocarbon group and a hydrocarbon group which is bonded by 2 or more of these groups. These hydrocarbon groups may is bonded by one or two or more of a univalent hydrocarbon group (an aliphatic hydrocarbon group, an alicyclic hydrocarbon group, an aromatic hydrocarbon group or a hydrocarbon group which is bonded by two or more of them). Further, the bivalent hydrocarbon includes a hydrocarbon group having a substituent, too. As the substituent, there may be mentioned the same groups as a substituent of the said ring Z¹.

As typical examples of the bivalent hydrocarbon, there may be mentioned, for example, an alkylene group such as a methylene, a methylmethylene, an ethylmethylene, a dimethylmethylene, an ethylmethylmethylene, an ethylene, a propylene, a trimethylene and a tetramethylene group; an alkenylene group such as a propenylene group; a cycloalkylene such as a 1,3-cyclopentylene, a 1,2-cyclohexylene, a 1,3-cyclohexylene and a 1,4-cyclohexylene group; a cycloalkylidene group such as a cyclopropylene, a cyclopentylidene and a cyclohexylidene group; an arylene group such as a phenylene group; a benzylidene group; a group wherein at least one hydrogen atom had by such groups is substituted by a fluorine atom; and others.

As a substituent of —NH— group, there may be mentioned an alkyl group (a alkyl group C₁ to C₄ of and others) such as a methyl and an ethyl group, an acyl group (a acyl group of C₁ to C₆ and others) such as an acetyl group, an alkoxycarbonyl group (a alkoxy-carbonyl group of C₁ to C₆ and others) such as a methoxycarbonyl group, and others. As a bivalent group which is bonded by plural numbers of bivalent hydrocarbon groups or others, there may be mentioned, for example, a group formed by bonding with a bivalent hydrocarbon group and an oxygen atom, a group formed by bonding 2 or more bivalent hydrocarbon group through an oxygen atom, an ester group, an amide group and others.

In the formula (1a), R is an unsaturated acyl group represented by the said formula (2). In the formula (2), each of R⁴, R⁵ and R⁶ is a hydrogen atom, a fluorine atom, an alkyl group or a fluoroalkyl group. As the alkyl group and the fluoroalkyl group, there may be mentioned a group exemplified in the said R¹ and R². As R⁴, a fluorine atom or a fluoroalkyl group of 1 to 3 carbon atoms is preferable, particularly a trifluoromethyl group is preferable. As each of R⁵ and R⁶, a hydrogen atom, an alkyl group of 1 to 3 carbon atoms or a fluoroalkyl group of 1 to 3 carbon atoms is preferable, particularly a hydrogen atom is preferable.

As typical examples of unsaturated acyl groups represented by the formula (2), a 2-trifluoromethylacryloyl-2-propenoyl group (=α-trifluoromethylacryloyl group), 2-fluoro-2-propenoyl group (=α-fluoroacryloyl group), an acryloyl group, a methacryloyl group and others. Further, in the formula (1a), at least one of R¹, R², R³, R⁴, R⁵ or R⁶ is a fluorine atom or a fluoroalkyl group.

In a fluorine-atom-containing-polymerizable unsaturated monomer represented by the formula (1a), when a fluorine atom bonds to a carbon atom of adjacent position (a position) to a carbon atom bonded by an RO— group, acid-eliminating ability is declined and therefore it is preferable that a fluorine atom isn't bonded to every carbon atom of adjacent position (a position) to a carbon atom bonded by an RO— group.

Further, as a preferable monomer in a fluorine-atom-containing-polymerizable unsaturated monomer represented by the formula (1a), there may be mentioned (i) a monomer bonded by a fluorine atom or a fluoroalkyl group (e.g., a C₁₋₁₀ fluoroalkyl group, particularly a fluoroalkyl group of C₁ to C₅) to the ring Z¹, (ii) a monomer having a fluoroalkyl group wherein at least one of R¹ or R² has a hydrogen atom at 1st position, (iii) a monomer having an alkyl group which may be fluorinated, wherein both of R¹ and R² have a hydrogen atom at 1st position, or (iv) a monomer wherein at least one of R¹ or R² is an alkyl group of 3 or more carbon atoms which may be fluorinated.

A polymeric compound having a repeated unit (a monomer unit) corresponding to the said monomer (i) bonded by a fluorine atom or a fluoroalkyl group to the ring Z¹ is superior in transparency for light of 300 nm or less wavelength, particularly vacuum ultraviolet light, and can improve sensitivity and resolution of resist. A number of fluorine atom or fluoroalkyl group may be one or two or more.

A polymeric compound having a repeated unit corresponding to the monomer (ii) wherein at least one of the said R¹ and R² a fluoroalkyl group having a hydrogen atom at 1st position is particularly superior in light-transparency and acid-elimination and thereby sensitivity and resolution of resist can be improved. As the fluoroalkyl group having a hydrogen atom at 1st position, there may be mentioned a linear- or branched-chain fluoroalkyl group having a hydrogen atom at 1st position in groups exemplified as the said fluoroalkyl group such as, for example, a 2,2,2-trifluoroethyl, a 2,2,2-trifluoro-1-(trifluoromethyl)ethyl, a 2,2,3,3,3-pentafluoropropyl, a 2,2,3,3-tetrafluoropropyl, a 2,2,3,3,4,4,4-heptafluorobutyl, a 2,2,3,3,4,4-hexafluorobutyl, a 2,2,3,3,4,4,5,5,5-nonafluoropentyl, a 2,2,3,3,4,4,5,5-octafluoropentyl, a 2,2,3,3,4,4,5,5,6,6,6-undecafluorohexyl, a 2,2,3,3,4,4,5,5,6,6-undecafluorohexyl, a 3,3,3,4,4,5,5,6,6,6-nonafluorohexyl, a 2,2,3,3,4,4,5,5,6,6,7,7,7-tridecafluoroheptyl, a 2,2,3,3,4,4,5,5,6,6,7,7-dodecafluoroheptyl, a 2,2,3,3,4,4,5,5,6,6,7,7,8,8,8-pentadecafluorooctyl, a 2,2,3,3,4,4,5,5,6,6,7,7,8,8,9,9,9-heptadecafluorononyl and a 2,2,3,3,4,4,5,5,6,6,7,7,8,8,9,9,10,10,10-nonadecafluorodecyl group. A number of carbon of the fluoroalkyl group is, for example, 2 to 15, preferably 2 to 12 and more preferably about 2 to 10. In a monomer of the said (ii), both of R¹ and R² may be a fluoroalkyl group having a hydrogen at 1st position. In the case, R¹ and R² may be a same group or a different group each other.

A polymeric compound having a repeated unit corresponding to the monomer (iii) wherein both of the said R¹ and R² are an alkyl group having a hydrogen atom at 1st position which may be fluorinated is extremely superior about acid-elimination and sensitivity and resolution of resist can be improved still more. As the fluoroalkyl group having a hydrogen atom at 1st position which may be fluorinated, there may be mentioned a linear- or branched-chain alkyl group (e.g., an alkyl group of 1 to 15 carbon atoms, preferably an alkyl group of 1 to 12 carbon atoms) and fluoroalkyl group (e.g., a fluoroalkyl group of 2 to 15 carbon atoms, preferably a fluoroalkyl group of 2 to 12 carbon atoms, more preferably a fluoroalkyl group of 2 to 10) having a hydrogen atom at 1st position in groups exemplified as the said alkyl group and fluoroalkyl group. In this case, R¹ and R² may be a same group or a different group each other.

A polymeric compound having a repeated unit corresponding to the monomer (iv) wherein at least one of the said R¹ and R² is an alkyl group of 3 or more carbon atoms (e.g., about 3 to 15, preferably about 3 to 10 and more preferably about 4 to 10) which may be fluorinated is particularly superior about acid-elimination and advantageous to sensitivity and resolution of resist. An alkyl group of 3 or more carbon atoms which may be fluorinated includes a group of 3 or more carbon atoms in groups exemplified as the said alkyl group and fluoroalkyl group. Both of R¹ and R² may be an alkyl group of 3 or more carbon atoms which may be fluorinated. In the case, R¹ and R² may be a same group or a different group each other.

As a typical example of fluorine-atom-containing polymerizable monomer represented by the formula (1a), the following compounds are exemplified. As a typical compound wherein the ring Z¹ is a ring represented by the formula (3a), there may be mentioned, for example, a compound wherein R⁴ in the formula (2) is a trifluoromethyl group such as 2-[1-(2-trifluoromethyl-2-propenyloxy)-1-methylethyl]norbornane, 2-[1-(2-trifluoromethyl-2-propenyloxy)-1,2-dimethylpropyl]norbornane, 2-[1-(2-trifluoromethyl-2-propenyloxy)-1-methylbutyl]norbornane, 2-[1-(2-trifluoromethyl-2-propenyloxy)-1-methylpentyl]norbornane, 2-[1-(2-trifluoromethyl-2-propenyloxy)-1-methylheptyl]norbornane, 2,3-bis(trifluoromethyl)-5-[1-(2-trifluoromethyl-2-prolenoyloxy)-1-methylpentyl]norbornane, 1,2,3,3,4,5,5,6,6,7,7-undecafluoro-2-[1-(2-trifuluoromethyl-2-propenoyloxy)-1-methylpentyl]norbornane, 2-[3,3,3-trifluoro-1-(2-trifluoromethyl-2-propenoyloxy)-1-methylpropyl]norbornane, 2-[3,3,4,4,5,5,6,6,7,7,7-undecafluoro-1-(2-trifluoromethyl-2-propenoyloxy)-1-methylheptyl]norbornane, 2-[3,3,4,4,5,5,6,6,7,7,8,8,9,9,9-pentadecafluoro-1-(2-trifluoromethyl-2-propenoyloxy)-1-methylnonyl]norbornane, 2-[3,3,4,4,5,5,6,6,7,7,8,8,9,9,10,10,10-heptadecafluoro-1-(2-trifluoromethyl-2-propenoyloxy)-1-methyldecyl]norbornane, 2-[3,3,3-trifluoro-1-(2,2,2-trifluoroethyl)-1-(2-trifluoromethyl-2-propenoyloxy)propyl]norbornane, 2-[3,3,3-trifluoro-1-(2,2,2-trifluoroethyl)-1-(2-trifluoromethyl-2-propenoyloxy)propyl]-2-trifluoromethylnorbornane, 3-[3,3,4,4,5,5,6,6,7,7,8,8,9,9,10,10,10-heptadecafluoro-1-(2-trifluoromethyl-2-propenoyloxy)-1-methyldecyl]-tetracyclo[4.4.0.1^{2,5}.1^{7,10}]dodecane and 2-[1-(2-trifluoromethyl-2-propenyoloxy)-1-methylpentyl]-7-oxabicyclo[2.2.1]heptane and a compound corresponding to the said compound wherein R⁴ is a fluorine atom and others.

As a typical compound wherein the ring Z¹ is a ring represented by the formula (3b), there may be mentioned, for example, a compound wherein R⁴ in the formula (2) is a trifluoromethyl group such as 1-[3,3,4,4,5,5,6,6,7,7,8,8,9,9,10,10,10-heptadecafluoro-1-(2-trifluoromethyl-2-propdnoyloxy)-1-methyldecyl]cyclohexane and 1-[3,3,4,4,5,5,6,6,7,7,8,8,9,9,10,10,10-heptadecafluoro-1-(2-trifluoromethyl-2-propdnoyloxy)-1-methyldecyl]-tricyclo [6.2.1.02,7]undecane and a compound corresponding to the said compound wherein R⁴ is a fluorine atom and others.

As a typical compound wherein the ring Z¹ is a ring represented by the formula (3c), there may be mentioned, for example, a compound wherein R⁴ in the formula (2) is a trifluoromethyl group such as 1-fluoro-3-[1-(2-trifluoromethyl-2-propenoyloxy)-1-methylethyl]adamantane, 1,3-difluoro-5-[1-(2-trifluoromethyl-2-propenoyloxy)-1-methylethyl]adamantane, 1,3,5-trifluoro-7-[1-(2-trifluoromethyl-2-propenoyloxy)-1-methylethyl]adamantane, 2,2,3,4,4,5,6,6,7,8,8,9,9,10,10-pentadacafluoro-1-[1-(2-trifluoromethyl-2-propenoyloxy)-1-methylethyl]adamantane, 1-[3,3,3-trifluoro-1-(2-trifluoromethyl-2-propenoyloxy)-1-methylpropyl]adamantane, 1-[3,3,4,4,5,5,6,6,7,7,8,8-dodecafluoro-1-(2-trifluoromethyl-2-propenoyloxy)propyl]adamantane and 1-[3,3,3-trifluoro-1-(2,2,2-trifluoroethyl)-1-(2-trifluoromethyl-2-propenoyloxy)propyl]adamantane and a compound corresponding to the said compounds wherein R⁴ is a fluorine atom.

In the said formula (1b), the ring Z² is an alicyclic hydrocarbon ring. As the alicyclic hydrocarbon ring, there may be mentioned the same as an alicyclic hydrocarbon ring in the said ring Z¹. An atom constituting the ring Z² may have a substituent. The substituent is the same as a substituent which may be had by an atom constituting the ring Z¹. Further, when a number of substituent of the ring Z² is 2 or more, they may bond each other to form 4 or more rings such as, for example, a cycloalkane ring and a lactone ring with a carbon atom constituting the ring Z². These rings may have a substituent such as a halogen atom which is a fluorine atom and others (the same substituent as a substituent which may be had by an atom constituting the said ring Z¹). R³ is an alkyl group or a fluoroalkyl group. As the alkyl group or the fluoroalkyl group, there may be mentioned the same substituent as a substituent exemplified in the said R¹ and R². R is the same as a case of the said formula (1a). As R⁴, a fluorine atom, a fluoroalkyl group of 1 to 3 carbon atoms, a hydrogen atom or an alkyl group of 1 to 3 carbon atoms is preferable, particularly a trifluoromethyl group is preferable. As each of R⁵ and R⁶, a hydrogen atom, an alkyl group of 1 to 3 carbon atoms or a fluoroalkyl group of 1 to 3 carbon atoms is preferable, particularly a hydrogen atom is preferable. In an adjacent position (e.g., 1st position of R³) of a carbon atom bonded by an RO— group represented by the formula (1b), there is a carbon atom bonded by at least one hydrocarbon.

In a fluorine-atom-containing polymerizable unsaturated-monomer presented by the formula (1b), when a fluorine atom bonds to a carbon atom of an adjacent position (α position) of a carbon atom bonded by an RO— group, acid-eliminating ability is declined and therefore it is preferable that a fluorine atom doesn't bond to any carbon atom of an adjacent position (α position) of a carbon atom bonded by an RO— group.

In the formula (1b), (i) a fluorine atom or fluoroalkyl group bonds to the ring Z², or (ii) R³ is a fluoroalkyl group (preferably, a fluoroalkyl group having a hydrogen atom at 1st position). Therefore, a polymeric compound having a repeated unit corresponding to a monomer represented by the formula (1b) is particularly superior to transparency for light of wavelength of 300 nm or less, especially vacuum ultraviolet light and thereby sensitivity and resolution of resist can be improved. In the case (i), a number of a fluorine atom or a fluoroalkyl group bonding to the ring Z² may be one or two or more. As a fluoroalkyl group of R³, there may be mentioned the same group as a fluoroalkyl group in the said R¹ and R².

As a typical example of a fluorine-atom-containing polymerizable unsaturated monomer represented by the formula (1b), the following compounds are exemplified. As a typical compound wherein the ring Z² is a ring represented by the formula (3a), there may be mentioned, for example, a compound wherein R⁴ of the formula (2) is a trifluoromethyl group such as 2-(2,2,2-trifluoroethyl)-2-(2-trifluoromethyl-2-propenoyloxy)norbornane, a 2-nonafluorobutyl-5-(2-trifluoromethyl-2-propenoyloxy)-5-methylnorbornane, 2-(3,3,4,4,5,5,6,6,6-nonafluorohexyl)-2-(2-trifluoromethyl-2-propenoyloxy)norbornane and 2,3,3,4,4,5,5,6-octafluoro-8-(2-trifluoromethyl-2-propenoyloxy)-8-methyltricyclo[5.2.1.0^{2,6}]decane and a compound corresponding to the said compound wherein R⁴ is a fluorine atom, a hydrogen atom or a methyl group.

As a typical compound wherein the ring Z² is a ring represented by the formula (3b), there may be mentioned, for example, a compound wherein R⁴ of the formula (2) is a trifluoromethyl group such as 1-(2,2,2-trifluoroethyl)-1-(2-trifluoromethyl-2-2propenoyl oxy)cyclohexane, 1-nanofluorobutyl-4-(2-trifluoromethyl-2-propenoyloxy)-4-m ethylcyclohexanone, 1-(3,3,4,4,5,5,6,6,6-nanofluorohexayl)-1-(2-trifluoromethyl-2-propenoyloxy)cyclohexane and 1,1,2,2,3,3,3a,7a-octafluoro-5-(2-trifluoromethyl-2-propen oyloxy)-5-methylperhydroindene and a compound corresponding to the said compound wherein R⁴ is a fluorine atom, a hydrogen atom or a methyl group.

As a typical compound wherein the ring Z² is a ring represented by the formula (3c), there may be mentioned, for example, a compound wherein R⁴ of the formula (2) is a trifluoromethyl group such as 2-(2,2,2-trifluoroethyl)-2-(2-trifluoromethyl-2-propenoyloxy)adamantane, 2-(3,3,4,4,5,5,6,6,6-nonafluorohexyl)-2-(2-trifluoromethyl-2-propenoyloxy)adamantane, 1-fluoro-4-(2-trifluoromethyl-2-propenoyloxy)-4-methyladamantane and 1,3-difluoro-6-(2-trfluoromethyl-2-propenoyloxy)-6-methyladamantane and a compound corresponding to the said compound wherein R⁴ is a fluorine atom, a hydrogen atom or a methyl group.

[Production of Fluorine-Atom-Containing Polymerizable Unsaturated-Monomer]

In a fluorine-atom-containing polymerizable unsaturated-monomer, a compound represented by the formula (1a) can be, for example, produced according to the following equation. 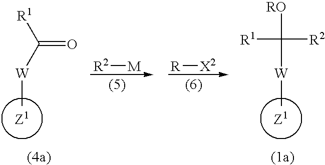
[wherein M is a metallic element or a —MgX¹ group (X¹ is a halogen atom). X² is a halogen atom. The ring Z¹, R, R¹, R² and W is the same as the above-mentioned one. Provided that, at least one of R¹, R², R¹, R⁵ and R⁶ is a fluorine atom or a fluoroalkyl group.]

Namely, a carbonyl compound represented by the formula (4a) and a (fluoro) alkylating agent represented by the formula (5) are reacted in a proper solvent such as tetrahydrofuran, then a resulting reaction product is reacted with an unsaturated carboxylic acid halide represented by the formula (6) and thereby a corresponding compound represented by the formula (1a) can be obtained. In the equation, as a metallic element of M, there may be mentioned lithium and others. The metallic element may have a ligand. A (fluoro) alkylating agent represented by the formula (5) may be generated in the reaction system. For example, a compound represented by R²—Li can be generated by reacting a compound represented by R²—X³ (X³ is a halogen atom) with methyl lithium in the system. In a compound represented by the formula (5), a compound wherein M is a Mg—X¹ group is generally named as a Grignard reagent. The Grignard reagent can be prepared by a common method. As a halogen atom of X¹, X² and X³, there may be mentioned a chlorine atom, a bromine atom, an iodine atom and others.

An amount used of each of a compound of the formula (5) and a compound of the formula (6) is about 1 to 1.5 mole based on 1 mole of a compound represented by the formula (4a). The reaction temperature is, varying according to a sort of material, about 30 to 120° C. The reaction product can be isolated and purified by a separating method such as an extraction, a washing, a concentration, a distillation, a crystallization, a recrystallization and a column chromatography.

A carbonyl compound represented by the formula (4a) which is used as a raw material in the above method can be obtained, for example, by applying a reaction described in Japan Patent Application Laid-Open No. 338514/2002. Namely, as represented by the following equation, an aldehyde represented by the formula (13) is reacted with an olefin represented by the formula (14) under an imide compound catalyst such as N-hydroxy phthalimide and thereby a carbonyl compound represented by the formula (15) which is a corresponding addition product can be obtained.

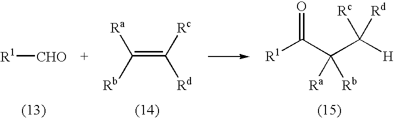

In the above reaction equation, R¹ is an alkyl group or a fluoroalkyl group as same as above and R^{a}, R^{b}, R^{c} and R^{d} are, identical to or different from, a hydrogen atom or an organic group. At least two of R^{a}, R^{b}, R^{c} and R^{d} may be bonded each other and thereby a ring may be formed with an adjacent carbon atom or a carbon-carbon bond. A group, “—C(R^{a})(R^{b})—CH(R^{c})(R^{d})”, of a compound represented by the formula (15) corresponds to a group, “—W-ring Z¹”, of a compound represented by the formula (4a). Therefore, for example, in carbonyl compounds represented by the formula (4a), a compound wherein W is a single bond and a ring Z¹ is a norbornane ring can be produced by reacting a R¹—CHO with a norbornene.

In the above reaction, making a radical generator such as benzoylperoxide to exist in the system is preferable. An using amount of the radical generator is, for example, usually about 0.001 to 0.5 mole based on 1 mole of a component of side being used less in both of a compound represented by the formula (13) and a compound represented by the formula (14). The reaction can be performed in a proper solvent such as an aromatic hydrocarbon a halogenated hydrocarbon and an ether or in no solvent. A ratio of a compound represented by the formula (13) and a compound represented by the formula (14) isn't particularly limited and the reaction may be performed either by equivalent mole or by excess amount of either one. A reaction temperature is usually about 40 to 125° C.

In a compound represented by the formula (1a), W is a single bond and a compound wherein Z¹ is an adamantane ring can be obtained, for example, by using a reaction described in a pamphlet of Patent international No 99/54271.

In addition, in a compound represented by the formula (1a), a compound (1a′) wherein each of R¹ and R² is identical can be produced by according to the following equation; 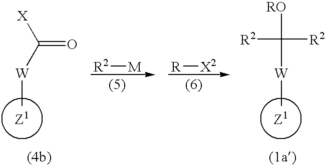
wherein X is a halogen atom, each of the ring Z¹, R, R², W, X² and M is identical to the above description and provided that at least one of R¹, R², R⁴, R⁵ and R⁶ is a fluorine atom or a fluoroalkyl group.

Namely, by reacting a carboxylic acid halide represented by the formula (4b) with a (fluoro) alkylating agent and then reacting the obtained reaction product with an unsaturated carboxylic acid halide, a corresponding compound represented by the formula (1a′) can be obtained. As a halogen atom in X, there may be mentioned a chlorine atom, a bromine atom, a iodine atom and others. An used amount of a compound of the formula (5) is 2 to 3 mole based on 1 mole of a compound represented by the formula (4b) and an used amount of a compound of the formula (6) is 1 to 1.5 mole based on 1 mole of a compound represented by the formula (4b). The reaction temperature is, varying according to a sort of material, about 30 to 120° C. The reaction product can be isolated and purified by a method such as an extraction, a washing, a concentration, a distillation, a crystallization, a recrystallization and a column chromatography. Further, a compound used as a raw material represented by the formula (4b) can be produced by reacting a corresponding carboxylic acid with a halogenating agent such as thionyl chloride. The reaction condition is identical to an usual halogenating reaction.

In a fluorine-atom-containing polymerizable unsaturated-monomer of the present invention, a compound represented by the formula (1b) can be produced, for example, by according to the following equation; 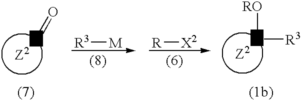
wherein each of the ring Z², R, R³, X³ and M is identical to the above description, and provided that (i) a fluorine atom or a fluoroalkyl group is bonded to the ring Z² or (ii) R³ is a fluoroalkyl group.
Namely, by allowing a cyclic ketone compound represented by the formula (7) to react with a (fluoro) alkylating agent represented by the formula (8) in a proper solvent such as tetrahydrofuran and then allowing the obtained reaction product to react with an unsaturated carboxylic acid halide represented by the formula (6), a corresponding compound represented by the formula (1b) can be obtained. Used amounts of a compound of the formula (8) and a compound of the formula (6) are respectively about 1 to 1.5 moles based on one mole of a compound represented by the formula (7). The reaction temperature is usually 30 to 120° C., varying according to a sort of raw material. The reaction product can be isolated and purified by a separating method such as an extraction, a washing, a concentration, a distillation, a crystallization, a recrystallization and a column chromatography. A carbonyl compound, represented by the formula (7), used as a raw material in the above production can be applied a manufactured product and can be produced by a known method or by using a known reaction.

A fluorine-atom-containing polymerizable unsaturated-monomer of the present invention represented by the formula (1a) or (1b) can be also produced by allowing an alcohol corresponding to a compound represented by the formula (1a) or (1b) [namely, a compound in which R in the formula (1a) or (1b) is a hydrogen atom] to react [an esterification] with an unsaturated carboxylic acid [namely, a compound represented by the formula C(R⁵) (R⁶)═C(R⁴)—COOH] corresponding to an saturated acyl group represented by the formula (2) or a reactive derivative thereof. As the said reactive derivative of unsaturated carboxylic acid, there may be mentioned an acid halide (an acid chloride, an acid bromide and others), an acid anhydride, an ester (a methyl ester, an ethyl ester and others) and so on. The esterification can be performed by following to a common esterification.

For example, in the above reaction, when an acid halide or an acid anhydride of the said unsaturated carboxylic acid is used to the reaction as one raw material, the reaction is usually performed under a base. As the base, for example, an organic base group such as triethylamine and pyridine and an inorganic base group such as sodium hydroxide, sodium carbonate and sodium bicarbonate. An used amount of base is usually about 1 to 1.5 equivalent amount based on an acid halide or an acid anhydride of unsaturated carboxylic acid and may be used in large excess. It is preferable that the reaction is performed in an inactive solvent for the reaction such as toluene, methylene chloride and tetrahydrofuran. An used amount of an acid halide or an acid anhydride of unsaturated carboxylic acid is usually about 0.9 to 1.3 moles based on one mole of an alcohol corresponding to a compound represented by the formula (1a) or (1b). the reaction temperature is usually about −10° C. to 100° C., varying according to a sort of raw material used to the reaction.

In the above reaction, when using an ester of the said unsaturated carboxylic acid to the reaction as one raw material, it is preferable that an ester exchanging catalyst is used as a catalyst. As the ester exchanging catalyst, a common one in an organic synthesis field such as a metal compound including an aluminium compound, a titan compound and others can be used. It is preferable that the reaction is performed in an inactive solvent for the reaction such as toluene. The reaction may be also performed while distilling out an alcohol by-produced by the reaction. An used amount of ester of unsaturated carboxylic acid is usually about 0.9 to 1.3 moles based on one mole of an alcohol corresponding to a compound represented by the formula (1a) or (1b). The reaction temperature can be, varying according to a sort of raw material, usually selected from a scope of about 20 to 150° C. properly. In all of the above methods, a reaction product can be isolated and purified by a separating method such as an extraction, a washing, a concentration, a distillation, a crystallization, a recrystallization and a column chromatography.

[Polymeric Compound]

A polymeric compound of the present invention contains a repeated unit (a monomer unit)corresponding to the said fluorine-atom-containing polymerizable unsaturated-monomer of the present invention. The said repeated unit may be one sort or may be two sorts or more. Such a polymeric compound can be produced by subjecting the said fluorine-atom-containing polymerizable unsaturated-monomer to a polymerization.

As a polymeric compound of the present invention has various functions required as a resist in sufficient balance, another repeated unit may be had in addition to a repeated unit corresponding to the above fluorine-atom-containing polymerizable unsaturated-monomer of the present invention. Such another repeated unit can be formed by allowing a polymerizable monomer corresponding to the said repeated unit to co-polymerize with the fluorine-atom-containing polymerizable unsaturated-monomer of the present invention. As the said another repeated unit, there may be mentioned, for example, a repeated unit having substrate adhesion and/or hydrophilicity function, a repeated unit improving acid-eliminating function, a repeated unit improving etching resistance, a repeated unit improving transparency and so on. The said hydrophilicity function includes a function improving solubility against a resist solvent and an alkali developer. Further, when preparing a polymeric compound of the present invention, a monomer which is used to perf orm co-polymerization smoothly or to unify a composition of co-polymer can be also applied.

A repeated unit having substrate adhesion and/or hydrophilicity function can be introduced into a polymer by using a polymerizable unsaturated monomer having a polar group as a co-monomer. As the said polar group, there may be mentioned, f or example, a hydroxyl group which may have a protecting group, a carboxyl group which may have a protecting group, an amino group which may have a protecting group, a sulfo group which may have a protecting group, a group having a lactone ring and so on. As the said protecting group, a common one in an organic synthesis field can be applied (for example, a protecting group exemplified above). As a polymerizable unsaturated monomer having a polar group, a known compound in a resist field can be used.

A repeated unit improving acid-eliminating function can be introduced into a polymer by using, for example, (1) a (meth)acrylic acid ester derivative bonded by a hydrocarbon group having a tertiary carbon at an adjacent position of an oxygen atom constituting an ester, a 2-tetrahydrofuranyl group, a 2-tetrahydropyranyl group or others, (2) a (meth) acrylic acid ester derivative having a hydrocarbon group (such as an alicyclic hydrocarbon group, an aliphatic hydrocarbon group and a group which these are bonded) at an adjacent position of an oxygen atom constituting an ester and in which the said hydrocarbon group is bonded by —COOR^{c} group (R^{c} is a tertiary hydrocarbon group, a 2-tetrahydrofuranyl group or a 2-tetrahydropyranyl group) directly or through a combining group as a co-monomer. As such a (meth)acrylic acid ester derivative, a known compound in a resist field can be used.

A repeated unit improving transparency can be introduced into a polymer by using, for example, a polymerizable monomer having a fluorine atom in the molecular as a co-monomer. As a typical example used to give various functions as a resist to a polymeric compound of the present invention except for a monomer of the present invention, there may be mentioned a vinyl ether monomer represented by the above formula (9a) or (9b). By allowing such a vinyl ether monomer, especially a vinyl ether monomer having an alicyclic hydrocarbon ring, to co-polymerize, etching resistance of the polymer can be improved. Further, by using a vinyl ether monomer having a polar group in the molecular, substrate adhesion and/or hydrophilicity can be improved. These vinyl ether monomers can be used alone or by combining two sorts or more.

In the formula (9a), a ring Z³ is a ring represented by the above formulae (10a) to (10i). In the formulae (10a) to (10i), Y⁴ is an alkylene group, an oxygen atom or a sulfur atom and each of Y⁵, Y⁶, Y⁷ and Y⁸ is an alkylene group, an oxygen atom, a sulfur atom or non-bonding. As an alkylene group, there may be mentioned, for example, a linear or branched chain alkylene group of carbon number 1 to 3 (preferably, 1 or 2) such as a methylene, an ethylene, a propylene and a trimethylene group and others. As Y⁴ and Y⁵, an alkylene group of carbon number 1 to 3 such as, particularly a methylene group, or an oxygen atom is preferable. As Y⁶, Y⁷ and Y⁸, an alkylene group of carbon number 1 to 3 such as, particularly a methylene group, an oxygen atom or non-bonding is preferable. Each of a, c, d and e denotes an integer of 0 to 3 and preferably 0 to 2, and particularly 0 or 1 is preferable. b denotes 1 or 2. In the formula, a ring may have a substituent. As the said substituent, there may be mentioned the same as a substituent which the said ring Z¹ may have.

W¹ is a bivalent hydrocarbon group. In the bivalent hydrocarbon group, a bivalent aliphatic hydrocarbon group, a bivalent alicyclic hydrocarbon group, a bivalent aromatic hydrocarbon group and a hydrocarbon group unified by two or more of these are included. These hydrocarbon group may be bonded by one or two or more univalent hydrocarbon groups (an aliphatic hydrocarbon group, an alicyclic hydrocarbon group, an aromatic hydrocarbon group and a hydrocarbon group unified by two or more of these). Further, in a bivalent hydrocarbon group, a hydrocarbon group having a substituent is included. As the substituent, for example, a halogen atom (such as a fluorine atom), an oxo group, a hydroxyl group, a substituted oxy group (for example, an alkoxy group, an aryloxy group, an aralkyloxy group, an acyloxy group and others), a carboxyl group, a substituted oxycarbonyl group (an alkoxycarbonyl group, an aryloxycarbonyl group, an aralkyloxycarbonyl group and so on), a substituted or unsubstituted carbamoyl group, a cyano group, a nitro group, a substituted or unsubstituted amino group, a sulfo group, a heterocyclic group and others may be had. The said hydroxyl group and carboxyl group may be protected by a protecting group which is common in an organic synthesis field. In addition, a ring of an alicyclic hydrocarbon group or an aromatic hydrocarbon group may be condensed by an aromatic or non-aromatic hetero ring.

As a typical example of a bivalent hydrocarbon group, there may be mentioned, for example, an alkylene group such as a methylene, a methylmethylene, an ethylmethylene, a dimethylmethylene, an ethylmethylmethylene, an ethylene, a propylene, a trimethylene, and a tetramethylene group; an alkenylene group such as a propenylene group; a cycloalkylene group such as a 1,3-cyclopentylene, a 1,2-cyclohexylene, a 1,3-cyclohexlene and a 1,4-cyclohexylene group; a cycloalkylidene group such as a cyclopropylene, a cyclopentylidene and a cyclohexylidene group; an arylene group such as a phenylene group; a benzylidene group; a group in which at least one hydrogen atom had by these groups is substituted by a fluorine atom and so on.

In a preferable example of W1, for example, a group represented by the following formula (16) is included (in the formula, each of R²¹ and R²² is identical to or different from a hydrogen atom or a hydrocarbon group. may be combined together to form an alicyclic ring with an adjacent carbon atom).

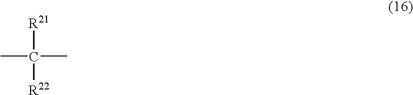

As a hydrocarbon group in R²¹ and R²², there may be mentioned analiphatic hydrocarbon group (such as an alkyl group of carbon number about 1 to 20, an alkenyl group of carbon number about 2 to 20, an alkynyl group of carbon number about 2 to 20), an alicyclic hydrocarbon group (such as a cycloalkyl group of about 3 to 20 members, a cycloalkenyl group of about 3 to 20 members a bridged cyclic hydrocarbon group), an aromatic hydrocarbon group (such as an aromatic hydrocarbon group of carbon number about 6 to 14) and a group in which two or more of these are unified. In the said hydrocarbon group, a hydrocarbon group having a substituent is included, too. As the said substituent, there may be mentioned the same as a substituent which a bivalent hydrocarbon of the said W¹ may have.

In preferable R²¹ and R²², a hydrogen atom; an alkyl group of C₁ to C₁₀ (particularly, an alkyl group of C₁ to C₅) such as a methyl, an ethyl, a propyl, an isopropyl and a butyl group; a cycloalkyl group which may have a substituent such as a cyclopentyl group and a cyclohexyl group; a bridged cyclic group which may have a substituent such as a norbornane-2-yl group and an adamantene-1-yl group; and others are included. As a substituent which a cycloalkyl group and a bridged cyclic group may have, there may be mentioned the same as a substituent which the said ring Z¹ may have.

In the formula (9a), each of R⁷, R⁸ and R⁹ is identical to or different from a hydrogen atom or an organic group. As an organic group in R⁷, R⁸ and R⁹, there may be mentioned, for example, a halogen atom, a hydrocarbon group, a heterocyclic group, a substituted oxycarbonyl group (such as an alkoxycarbonyl group, an aryloxycarbonyl group, an aralkyloxycarbonyl group and a cycloalkyloxycarbonyl group), a carboxyl group, a substituted or unsubstituted carbamoyl group, a cyano group, a nitro group, a sulfur acid group, a sulfur acid ester group, an acyl group (an aliphatic acyl group such as an acetyl group; an aromatic acyl group such as a benzoyl group and others), an alkoxy group (an alkoxy group of C₁ to C₆ such as a methoxy group and an ethoxy group, and others), a N,N-di substituted amino group (such as a N,N-dimethyl amino group and a piperidino group), a group unified by two or more of these and others, and the said carboxyl group and others may be protected by a protecting group which is common in an organic synthesis field. As the said halogen atom, there may be mentioned a fluorine atom, a chlorine atom, a bromine atom and an iodine atom. In these organic groups, a hydrocarbon group, a hetero cyclic group and others are preferable.

The said hydrocarbon group and heterocyclic group include a hydrocarbon group and heterocyclic group having a substituent, too. As a hydrocarbon group, there may be mentioned the same as a hydrocarbon group in the said R²¹ and R²². In a preferable hydrocarbon group, an alkyl group of C₁ to C₁₀, an alkenyl group of C₂ to C₁₀, an alkynyl group of C₂ to C₁₀, a cycloalkyl group of C₃ to C₁₅, an aromatic hydrocarbon group of C₆ to C₁₀, a C₃ to C₁₀ cycloalkyl-C₁ to C₄ alkyl group, an aralkyl group of C₇ to C₁₄ and others are included. These hydrocarbon groups may have a substituent and as the said substituent, there may be mentioned the same as a substituent which a bivalent hydrocarbon group of the said W¹ may have.

In a hetero ring constituting a heterocyclic group of the said R⁷ and others, an aromatic hetero ring and a non-aromatic hetero ring are included. As such a hetero ring, there may be mentioned, for example, a hetero ring having an oxygen atom, a sulfur atom and a nitrogen atom as a hetero atom, and so on. The heterocyclic group may have, in addition to a substituent which the said hydrocarbon group may have, a substituent such as an alkyl group (for example, an alkyl group such as a methyl and an ethyl group, and others), a cycloalkyl group, an aryl group (for example, a phenyl, a naphtyl group and others).

In preferable R⁷, R⁸ and R⁹, a hydrogen atom, a hydrocarbon group (for example, an alkyl group of C₁ to C₁₀, an alkenyl group of C₂ to C₁₀, an alkynyl group of C₂ to C₁₀, a cycloalkyl group of C₃ to C₁₅, an aromatic hydrocarbon group of C₆ to C₁₀, a C₃ to C₁₀ cycloalkyl-C₁ to C₄ alkyl group, an aralkyl group of C₇ to C₁₄ and others are included. As R⁷, R⁸ and R⁹, a hydrogen atom and an alkyl group of C₁ to C₃ such as a methyl group are particularly preferable.

In the formula (9a), at least two of Z³, W¹, R⁷, R⁸ and R⁹, are bonded together to form a ring with one or two or more atoms which are adjacent. In such a ring, a non-aromatic carbon ring or hetero ring is included. In the formula (9a), p denotes 0 or 1. q denotes an integer of 1 to 8, preferably an integer of 1 to 4, more preferably an integer of 1 to 3. When q is 2 or more, each of groups in inside of q pairs of brackets may be identical to or different from.

In a typical example of a vinyl ether compound represented by the formula (9a), the following compounds are included. As a vinyl ether compound in which a ring Z³ is a ring represented by the formula (10a), there may be mentioned, for example, 2-vinyloxynorbornane, 5-methoxycarbonyl-2-vinyloxynorbornane, 2-[1-(norbornane-2-yl)-1-vinyloxyethyl]norbornane, 2-(vinyloxymethyl)norbornane, 2-(1-methyl-1-vinyloxyethyl)norbornane, 2-(1-methyl-1-vinyloxypentyl)norbornane, 3-hydroxy-4-vinyloxytetracyclo[4.4.0.1^{2,5}.1^{7,10}]dodecane, 3-hydroxy-8-vinyloxytetracyclo[4.4.0.1^{2,5}.1^{7,10}]dodecane, 3-methoxycarbonyl-8-vinyloxytetracyclo[4.4.0.1^{2,5}.1^{7,10}]dodecane, 3-methoxycarbonyl-9-vinyloxytetracyclo[4.4.0.1^{2,5}.1^{7,10}]dodecane, 3-(vinyloxymethyl)tetracyclo[4.4.0.1^{2,5}.1^{7,10}]dodecane, 3-hydroxymethyl-8-vinyloxytetracyclo[4.4.0.1^{2,5}.1^{7,10}]dodecane, 3-hydroxymethyl-9-vinyloxytetracyclo[4.4.0.1^{2,5}.1^{7,10}]dodecane, 8-hydroxy-3-(vinyloxymethyl)tetracyclo[4.4.0.1^{2,5}.1^{7,10}]dodecane, 9-hydroxy-3-(vinyloxymethyl)tetracyclo[4.4.0.1^{2,5}.1^{7,10}]dodecane, an isopropenyl group corresponding to these and others.

As a vinyl ether compound in which a ring Z³ is a ring represented by the formula (10b), there may be mentioned, for example, vinyloxycyclopentane, vinyloxycyclohexane, cis-1,1,3-trimethyl-5-vinyloxycyclohexane, trans-1,1,3-trimethyl-5-vinyloxycyclohexane, 1-isopropyl-4-methyl-2-vinyloxycyclohexane, 3-vinyloxytricyclo[6.2.1.0^{2,7}]undecane, 4-vinyloxytricyclo[6.2.1.0^{2,7}]undecane, 2-vinyloxy-7-oxabicyclo[3.2.1]octane-6-on, (2,2,2-trifluoro-1-trifluoromethyl-1-cyclohexylethyl)vinyl ether, [2,2,2-trifluoro-1-trifluoromethyl-1-(4-trifluoromethylhexyl)ethyl]vinyl ether, (1-trifluoromethyl-1-cyclohexyl)ethyl)vinyl ether, [1-trifluoromethyl-1-(4-trifluoromethylcyclohexyl)ethyl]vi nyl ether, and so on.

As a vinyl ether compound in which a ring Z³ is a ring represented by the formula (10c), there may be mentioned, for example, 1-vinyloxyadamantane, 2-vinyloxyadamantane, 2-methyl-2-vinyloxyadamantane, 2-ethyl-2-vinyloxyadamantane, 1-hydroxy-3-vinyloxyadamantane, 1,3-dihydroxy-5-vinyloxyadamantane, 1,3,5-trihydroxy-7-vinyloxyadamantane, 1,3-dimethyl-5-vinyloxyadamantane, 1-hydroxy-3,5-dimethyl-7-vinyloxyadamantane, 1-carboxy-3-vinyloxyadamantane, 1-amino-3-vinyloxyadamantane, 1-nitro-3-vinyloxyadamantane, 1-sulfo-3-vinyloxyadamantane, 1-sulfo-3-vinyloxyadamantane, 1-t-butyloxycarbonyl-3-vinyloxyadamantane, 4-oxo-1-vinyloxyadamantane, 1-(vinyloxymethyl)adamantane, 1-(1-methyl-1-vinyloxyethyl)adamantane, 1-(1-ethyl-1-vinyloxyethyl)adamantane, 1-(1-(norbornane-2-yl)-1-vinyloxyethyl)adamantane, an isopropenyl group corresponding to these and others.

As a vinyl ether compound in which a ring Z³ is a ring represented by the formula (10d), there may be mentioned, for example, 8-vinyloxy-4-oxatricyclo[5.2.1.0^{2,6}]decane-3,5-dion, 4-vinyloxy-11-oxapentacyclo[6.5.1.1^{3,6}.0^{2,7}.0^{9,13}]pentadecane-10,12-dion, an isopropenyl group corresponding to these and others.

As a vinyl ether compound in which a ring Z³ is a ring represented by the formula (10e), there may be mentioned, for example, α-vinyloxy-γ-butyrolactone, β-vinyloxy-γ-butyrolactone, γ-vinyloxy-γ-butyrolactone, α-vinyloxy-γ, γ-dimethyl-γ-butyrolactone, α,γ,γ-trimethyl-α-vinyloxy-γ-butyrolactone, γ,γ-dimethyl-β-methoxycarbonyl-α-vinyloxy-γ-butyrolactone, 8-vinyloxy-4-oxatricyclo[5.2.1.0^{2,6}]decane-3-on, 9-vinyloxy-4-oxatricyclo[5.2.1.0^{2,6}]decane-3-on, an isopropenyl group corresponding to these and others.

As a vinyl ether compound in which a ring Z³ is a ring represented by the formula (10f), there may be mentioned, for example, 4-vinyloxy-2,7-dioxabicyclo[3.3.0]octane-3,6-dion.

As a vinyl ether compound in which a ring Z³ is a ring represented by the formula (10g), there may be mentioned, for example, 5-vinyloxy-3-oxatricyclo[4.2.1.0^{4,8}]nonane-2-on, 5-methyl-5-vinyloxy-3-oxatricyclo[4.2.1.0^{4,8}]nonane-2-on, 9-methyl-5-vinyloxy-3-oxatricyclo[4.2.1.0^{4,8}]nonane-2-on, an isopropenyl group corresponding to these and others.

As a vinyl ether compound in which a ring Z³ is a ring represented by the formula (10h), there may be mentioned, for example, 6-vinyloxy-3-oxatricyclo[4.3.1.1^{4,8}]undecane-2-on, 6-hydroxy-8-vinyloxy-3-oxatricyclo[4.3.1.1^{4,8}]undecane-2-on, 8-hydroxy-6-vinyloxy-3-oxatricyclo[4.3.1.1^{4,8}]undecane-2-on, an isopropenyl group corresponding to these and others.

As a vinyl ether compound in which a ring Z³ is a ring represented by the formula (10i), there may be mentioned, for example, 1,4:3,6-dianhydro-D-glycidol 2(or 5)-vinyl ether, 1,4:3,6-dianhydro-L-glycidol 2(or 5)-vinyl ether, 1,4:3,6-dianhydro-D-manitol monovinyl ether, 1,4:3,6-dianhydro-L-manitol monovinyl ether, 1,4:3,6-dianhydro-D-iditol monovinyl ether, 1,4:3,6-dianhydro-L-iditol monovinyl ether, and others.

In the above formula (9b), R¹⁰ is an alkyl group which may have a substituent. As the alkyl group, there may be mentioned a linear or branched chain alkyl group of carbon number about 1 to 20 (preferably, carbon number 1 to 10) such as a methyl, an ethyl, a propyl, an isopropyl, a butyl, an isobutyl, a s-butyl, a t-butyl, a pentyl, a neopentyl, a hexyl, an octyl and a decyl group. As a substituent which the alkyl group may have, a halogen atom (such as a fluorine atom, a chlorine atom, a bromine atom, an iodine atom), an oxo group, a hydroxy group, a saturated oxy group (such as, for example, an alkoxy group, an aryloxy group, an aralkyloxy group and a acyloxy group), a carboxyl group, a saturated oxycarbonyl group (such as an alkoxycarbonyl group, an aryloxycarbonyl group and an aralkyloxycarbonyl group), a substituted or unsubstituted carbamoyl group, a cyano group, a nitro group, a substituted or unsubstituted amino group, a sulfo group, an aromatic hydrocarbon group, a heterocyclic group and others may be had. The said hydroxyl group or a carboxyl group may be protected by a protecting group which is common in an organic synthesis field.

Each of R¹¹, R¹² and R¹³ is identical to or different from a hydrogen atom or an organic group. As the organic group in R¹¹, R¹² and R¹³, there may be mentioned the same as an organic group in R⁷, R⁸ and R⁹.

In a preferable R¹¹, R¹² and R¹³, a hydrogen atom, a hydrocarbon group (an alkyl group of C₁ to C₁₀, an alkenyl group of C₂ to C₁₀, an alkynyl group of C₂ to C₁₀, a cycloalkyl group of C₃ to C₁₅, an aromatic hydrocarbon group of C₆ to C₁₀, a C₃ to C₁₀ cycloalkyl-C₁ to C₄ alkyl group, an aralkyl group of C₇ to C₁₄ and others) and others are included. As R¹¹, R¹² and R¹³, a hydrogen atom and an alkyl group of C₁ to C₃ such as a methyl group is particularly preferable.

In the formula (9b), at least two of R¹⁰, R¹¹, R¹² and R¹³, are bonded together to form a ring with one or two or more atoms which are adjacent. In such a ring, a non-aromatic carbon ring or hetero ring is included. In the formula (9b), r denotes an integer of 1 to 8, preferably an integer of 1 to 4, more preferably an integer of 1 to 3. When r is 2 or more, each of groups in inside of r pairs of brackets may be identical to or different from.

As a typical example of vinyl ether compound represented by the formula (9b), there may be mentioned, for example, methyl vinyl ether, ethyl vinyl ether, propyl vinyl ether, isopropyl s-butyl vinyl ether, t-butyl vinyl ether, hexyl vinyl ether, ethyleneglycol vinyl ether, ethyleneglycol divinyl ether, trifluoromethyl vinyl ether, (2,2,3,3-trifluotroethyl) vinyl ether, (2,2,3,3,3-pentafluoropropyl) vinyl ether, (2,2,3,3-tetrafluoropropyl) vinyl ether, (2,2,3,3,4,4,4-heptafluorobutyl) vinyl ether, (2,2,3,3,4,4-hexafluorobutyl) vinyl ether, (2,2,3,3,4,4,5,5,5-nonafluoropentyl) vinyl ether, (2,2,3,3,4,4,5,5-octafluoropentyl) vinyl ether, (2,2,3,3,4,4,5,5,6,6,7,7,7-tridecafluoroheptyl) vinyl ether, (2,2,3,3,4,4,5,5,6,6,7,7-dodecafluoroheptyl) vinyl ether, (2,2,3,3,4,4,5,5,6,6,7,7,8,8,9,9,10,10,10-heptadecafluorodecyl) vinyl ether, (2,2,3,3,4,4,5,5,6,6,7,7,8,8,9,9,10,10,11,11-icosadecafluoroundecyl) vinyl ether, (2,2,2-trifluoro-1-trifluoromethylethyl) vinyl ether, (1,2,2,2-tetrafluoro-1-trifluoromethylethyl) vinyl ether, [1,1-bis(trifluoromethyl)ethyl] vinyl ether, (1-trifluoromethyl-1-methylethyl) vinyl ether, (2,2,2-trifluoro-1-trifluoromethyl-1-phenylethyl) vinyl ether, (1-trifluoromethyl-1-phenylethyl) vinyl ether, an isopropenyl group corresponding to these, dihydropyran and others.

In the said vinyl ether monomer, (1) a compound in which a ring Z³ of a compound represented by the formula (9a) is a ring represented by the formula (10d), (10e), (10f), (10g), (10h) or (10i), (2) a compound in which a ring Z³ of a compound represented by the formula (9a) is a ring represented by the formula (10a), (10b) or (10c) and in which a ring Z³ is bonded by the said polar group or a group having the polar group or one of W1, R7, R8 and R9 is a group having a polar group and (3) a compound in which R10, R11, R12 or R13 of a compound represented by the formula (9b) is a group having a polar group are subjected as co-monomer to co-polymerization to form a repeated unit having substrate adhesion and/or hydrophilicity function.

The said vinyl ether monomer can be produced by a known method or by using a know reaction. Further, the vinyl ether monomer can be also obtained by allowing a vinyl ester such as vinyl acetate and vinyl propionate to react with a hydroxy compound corresponding to a portion except for a vinyl group in a required vinyl ether under an iridium compound catalyst and preferably under a base to convert the hydroxy compound to a vinyl ether. For example, 2-vinyloxy norbornane can be produced from vinyl acetate and 2-hydroxy norbornane. This reaction is performed under a solvent or without a solvent. An used amount of vinyl ether is, for example, about 0.1 to 5 mol based on one mol of a hydroxy compound. As an iridium compound catalyst, for example, an organic iridium compound catalyst such as di-μ-chlorobis(1,5-cyclooctadiene)II iridium (I) [Ir(cod)Cl]²) can be used. An used amount of iridium compound catalyst is, for example, 0.0001 to 1 mol based on one mol of hydroxy compound, preferably about 0.001 to 0.3 mol. As the said base, an inorganic base such as sodium carbonate can be used. An used amount of base is, for example, 0.001 to 3 mol based on one mol of hydroxy compound, preferably about 0.005 to 2 mol. The reaction temperature is can be properly selected according to a sort of reaction component and is, for example, about 50 to 150° C. A reaction product can be separate and purified by a separating method such as filtration, concentration, distillation, extraction, crystallization, re-crystallization and column chromatography.

As another typical example of polymerizable monomer except for a monomer of the present invention used to give various functions as resist to a polymeric compound of the present invention, there may be mentioned an acryl monomer [provided that except for a compound represented by the formulae (1a) and (1b)] presented by the above formula (11a), (11b), (11c) or (11d). Due to such an acryl monomer, when, for example, a fluorine atom is had in the molecule (for example, at acryl portion), transparency against a light of wavelength 300 nm or less, particularly a vacuum ultraviolet light can be improved, and when using an acryl monomer having an alicyclic carbon ring, etching resistance of a polymer can be improved, and when using an acryl monomer having a polar group in the molecule, substrate adhesion and/or hydrophilicity can be improved. In addition, when using an acryl monomer in which a tertiary carbon atom is bonded to an oxygen atom constituting an ester bond, acid-eliminating function can be improved. These acryl monomers can be used alone or by unified two sorts or more.

In the formula (11a), a ring Z⁴ is a cyclic group of the above formulae (10a) to (10i). In the formula (11a), W² is a bivalent hydrocarbon group. As the bivalent hydrocarbon group, there may be mentioned the same as a bivalent hydrocarbon group in the said W¹. Provided that both of a ring Z⁴ and W² don't have a fluorine atom and a group having a fluorine atom as a substituent. Each of R¹⁴, R¹⁵ and R¹⁶ is a hydrocarbon atom, a fluorine atom, an alkyl group or a fluoroalkyl group. Each of the alkyl group or the fluoroalkyl group is the same as an alkyl group and a fluoroalkyl group in R⁵ and R⁶. s denotes 0 or 1. t denotes an integer of 1 to 8, preferably an integer of 1 to 4, more preferably an integer of 1 to 3. When t is 2 or more, a group in t pairs of brackets may be identical to or different from.

As a typical example of acryl monomer represented by the formula (11a), there may be mentioned following one. As a compound in which a ring Z⁴ is a ring represented by the formula (10a), there may be mentioned, for example, a compound in which R¹⁴ is a trifluoromethyl group such as 2-(2-trifluoromethyl-2-propenoyloxy)norbornane, 3-hydroxy-4-(2-trifluoromethyl-2-propenoyloxy)tetracyclo[4.4.0.1^{2,5}.1^{7,10}]dodecane, 3-hydroxy-8-(2-trifluoromethyl-2-propenoyloxy)tetracyclo[4.4.0.1^{2,5}1^{7,10}]dodecane, 3-hydroxymethyl-8-(2-trifluoromethyl-2-propenoyloxy)tetracyclo[4.4.0.1^{2,5}.1^{7,10}]dodecane, 3-hydroxymethyl-9-(2-trifluoromethyl-2-propenoyloxy)tetracyclo[4.4.0.1^{2,5}.1^{7,10}]dodecane, 8-hydroxy-3-(2-trifluoromethyl-2-propenoyloxy)tetracyclo[4.4.0.1^{2,5}.1^{7,10}]dodecane, 9-hydroxy-3-(2-trifluoromethyl-2-propenoyloxy)tetracyclo[4.4.0.1^{2,5}.1^{7,10}]dodecane, a compound corresponding to the said compound in which R¹⁴ is a fluorine atom, a hydrogen atom or a methyl group, and others.

As a compound in which a ring Z⁴ is a ring represented by the formula (10b), there may be mentioned, for example, a compound in which R¹⁴ is a trifluoromethyl group such as 1-(2-trifluoromethyl-2-propenoyloxy)cyclopentane, 1-(2-trifluoromethyl-2-propenoyloxy)cyclohexane, cis-5-(2-trifluoromethyl-2-propenoyloxy)-1,1,3-trimethylcyclohexane, trans-5-(2-trifluoromethyl-2-propenoyloxy)-1,1,3-trimethyl cyclohexane, 2-(2-trifluoromethyl-2-propenoyloxy)-1-isopropyl-4-methylcyclohexane, 3-(2-trifluoromethyl-2-propenoyloxy)tricyclo[6.2.1.0^{2,7}]undecane, 4-(2-trifluoromethyl-2-propenoyloxy)tricyclo[6.2.1.0^{2,7}]undecane, 2-(2-trifluoromethyl-2-propenoyloxy)-7-oxabicyclo[3.2.1]octane-6-on, a compound corresponding to the said compound in which R¹⁴ is a fluorine atom, a hydrogen atom or a methyl group, and others.

As a compound in which a ring Z⁴ is a ring represented by the formula (10c), there may be mentioned, for example, a compound in which R¹⁴ is a trifluoromethyl group such as 1-(2-trifluoromethyl-2-propenoyloxy)adamantane, 2-(2-trifluoromethyl-2-propenoyloxy)adamantane, 2-(2-trifluoromethyl-2-propenoyloxy)-2-methyladamantane, 2-ethyl-2-(2-trifluoromethyl-2-propenoyloxy)adamantane, 1-(2-trifluoromethyl-2-propenoyloxy)-3-hydroxyadamantane, 1-(2-trifluoromethyl-2-propenoyloxy)-3,5-dihydroxyadamantane, 1-(2-trifluoromethyl-2-propenoyloxy)-3,5,7-trihydroxyadamantane, 5-(2-trifluoromethyl-2-propenoyloxy)-1,3-dimethyladamantane, 1-(2-trifluoromethyl-2-propenoyloxy)-3-hydroxy-5,7-dimethyladamantane, 1-carboxy-3-(2-trifluoromethyl-2-propenoyloxy)adamantane, 1-amino-3-(2-trifluoromethyl-2-propenoyloxy)adamantane, 3-(2-trifluoromethyl-2-propenoyloxy)-1-nitroadamantane, 3-(2-trifluoromethyl-2-propenoyloxy)-1-sulfoadamantane, 1-t-butyloxycarbonyl-3-(2-trifluoromethyl-2-propenoyloxy)a damantane, 1-(2-trifluoromethyl-2-propenoyloxy)-4-oxoadamantane, 1-[1-(2-trifluoromethyl-2-propenoyloxy)-1-methyethyl]adamantane, 1-[1-(2-trifluoromethyl-2-propenoyloxy)ethyl]adamantane, a compound corresponding to the said compound in which R¹⁴ is a fluorine atom, a hydrogen atom or a methyl group, and others.

As a compound in which a ring Z⁴ is a ring represented by the formula (10d), there may be mentioned, for example, a compound in which R¹⁴ is a trifluoromethyl group such as 8-(2-trifluoromethyl-2-propenoyloxy)-4-oxatricyclo[5.2.1.0 ^{2,6}]decane3,5-dion, 4-(2-trifluoromethyl-2-propenoyloxy)-11-oxapentacyclo[6.5.1.1^{3,6}.0^{2,7}.0^{9,13}]pentadecane-10,12-dion, a compound corresponding to the said compound in which R¹⁴ is a fluorine atom, a hydrogen atom or a methyl group, and others.

As a compound in which a ring Z ⁴ is a ring represented by the formula (10e), there may be mentioned, for example, a compound in which R¹⁴ is a trifluoromethyl group such as α-(2-trifluromethyl-2-propenoyloxy)-γ-butyrolactone, β-(2-trifluromethyl-2-propenoyloxy)-γ-butyrolactone, γ-(2-trifluromethyl-2-propenoyloxy)-γ-butyrolactone, α-(2-trifluromethyl-2-propenoyloxy)-γ, γ-dimethyl-γ-butyrolactone, α-(2-trifluromethyl-2-propenoyloxy)-α,γ,γ-dimethyl-γ-butyrolactone, 8-(2-trifluromethyl-2-propenoyloxy)-4-oxatricyclo[5.2.1.0^{2,6}]decane-3-on, 8-(2-trifluromethyl-2-propenoyloxy)-4-oxatricyclo[5.2.1.0^{2,6}]decane-3-on, a compound corresponding to the said compound in which R¹⁴ is a fluorine atom, a hydrogen atom or a methyl group, and others.

As a compound in which a ring Z⁴ is a ring represented by the formula (10f), there may be mentioned, for example, a compound in which R¹⁴ is a trifluoromethyl group such as 4-(2-trifluromethyl-2-propenoyloxy)-2,7-dion, a compound corresponding to the said compound in which R¹⁴ is a fluorine atom, a hydrogen atom or a methyl group, and others.

As a compound in which a ring Z⁴ is a ring represented by the formula (10 g), there may be mentioned, for example, a compound in which R¹⁴ is a trifluoromethyl group such as 5-(2-trifluromethyl-2-propenoyloxy)-3-oxatricyclo[4.2.1.0^{4,8}]nonane-2-on, 5-methyl-5-(2-trifluromethyl-2-propenoyloxy)-3-oxatricyclo [4.2.1.0⁴¹⁸]nonane-2-on, 9-methyl-5-(2-trifluromethyl-2-propenoyloxy)-3-oxatricyclo [4.2.1.0^{4,8}]nonane-2-on, a compound corresponding to the said compound in which R¹⁴ is a fluorine atom, a hydrogen atom or a methyl group, and others.

As a compound in which a ring Z⁴ is a ring represented by the formula (10h), there may be mentioned, for example, a compound in which R¹⁴ is a trifluoromethyl group such as 6-(2-trifluromethyl-2-propenoyloxy)-3-oxatricyclo[4.3.1.1^{4,8}]undecane-2-on, 8-(2-trifluromethyl-2-propenoyloxy)-6-hydroxy-3-oxatricyclo[4.3.1.1^{4,8}]undecane-2-on, 6-(2-trifluromethyl-2-propenoyloxy)-8-hydroxy-3-oxatricyclo[4.3.1.1^{4,8}]undecane-2-on, a compound corresponding to the said compound in which R¹⁴ is a fluorine atom, a hydrogen atom or a methyl group, and others.

As a compound in which a ring Z⁴ is a ring represented by the formula (10i), there may be mentioned, for example, a compound in which R¹⁴ is a trifluoromethyl group such as 1,4:3,6-dianhydro-D-glycidol 2(or 5)-(2-trifluromethyl-2-propenoyloxy), 1,4:3,6-dianhydro-L-glycidol 2(or 5)-(2-trifluromethyl-2-propenoyloxy), 1,4:3,6-dianhydro-D-manitol mono(2-trifluromethyl-2-propenoyloxy), 1,4:3,6-dianhydro-L-manitol mono(2-trifluromethyl-2-propenoyloxy), 1,4:3,6-dianhydro-D-initol mono(2-trifluromethyl-2-propenoyloxy), 1,4:3,6-dianhydro-D-iditol mono(2-trifluromethyl-2-propenoyloxy), a compound corresponding to the said compound in which R¹⁴ is a fluorine atom, a hydrogen atom or a methyl group, and others.

In the formula (11b), R¹⁷ is an alkyl group which may have a substituent. The said alkyl group which may have a substituent is the same as an alkyl group of the said R¹⁰ which may have a substituent. Each of R¹⁴, R¹⁵ and R¹⁶ is the same as above. u denotes an integer of 1 to 8, preferably an integer of 1 to 4, more preferably an integer of 1 to 3. When u is 2 or more, a group of inside of u pairs of brackets may be identical to or different from.

As a typical example of an acryl monomer represented by the formula (11b), there may be mentioned, for example, 2-trifouromethyl-2-methylpropenoate, 2-trifouromethyl-2-ethylpropenoate, 2-trifouromethyl-2-propylpropenoate, 2-trifouromethyl-2-isopropylpropenoate, 2-trifouromethyl-2-s-butylpropenoate, 2-trifouromethyl-2-t-butylpropenoate, 2-trifouromethyl-2-hexylpropenoate, 2-trifouromethyl-2-(2-hydroxyethyl)propenoate, 2-trifouromethyl-2-trifluoromethylpropenoate, 2-trifouromethyl-2-(2,2,2-trifluoroethyl)propenoate, 2-trifouromethyl-2-(2,2,3,3,3-pentafluoropropyl)propenoate, 2-trifouromethyl-2-(2,2,3,3-tetrafluoropropyl)propenoate, 2-trifouromethyl-2-(2,2,3,3,4,4,4-heptafluorobutyl)propenoate, 2-trifouromethyl-2-(2,2,3,3,4,4-hexafluorobutyl)propenoate, 2-trifouromethyl-2-(2,2,3,3,4,4,5,5,5-nonafluoropentyl)propenoate, 2-trifouromethyl-2-(2,2,3,3,4,4,5,5-octafluoropentyl)propenoate, 2-trifouromethyl-2-(2,2,3,3,4,4,5,5,6,6,7,7,7-tridecafluoroheptyl)propenoate, 2-trifouromethyl-2-(2,2,3,3,4,4,5,5,6,6,7,7-dodecafluoroheptyl)propenoate, 2-trifouromethyl-2-(2,2,3,3,4,4,5,5,6,6,7,7,8,8,9,9,10,10,10-heptadecafluorodecyl)propenoate, 2-trifouromethyl-2-(2,2,3,3,4,4,5,5,6,6,7,7,8,8,9,9,10,10,11,11,11-icosafluoroundecyl)propenoate, 2-trifouromethyl-2-(2,2,2-trifluoro-1-trifluoromethylethyl)propenoate, 2-trifouromethyl-2-(1,2,2,2-tetrafluoro-1-trifluoromethylethyl)propenoate, 2-trifouromethyl-2-[1,1-bis(trifluoromethyl)ethyl]propenoate, 2-trifouromethyl-2-(1-trifluoromethyl-1-methyethyl)propenoate, 2-trifouromethyl-2-(2,2,2-trifluoro-1-trifluoromethyl-1-phenylethyl)propenoate, 2-trifouromethyl-2-(1-trifluoromethyl-1-phenylethyl)propen oate, a compound corresponding to the said compound in which R¹⁴ is a fluorine atom, a hydrogen atom or a methyl group, and others.

In the formula (11c), a ring Z⁵ is an alicyclic carbon ring which may have a substituent. As the alicyclic carbon ring, there may be mentioned a ring represented the above formula (10a), (10b) or (10c) and so on. As a substituent which the alicyclic carbon ring may have there may be mentioned the same as a substituent which the said ring Z¹ may have. R¹⁸ is a hydrogen atom, a fluorine atom, an alkyl group or a fluoroalkyl group. The said alkyl group and fluoroalkyl group are same as an alkyl group and fluoroalkyl group in R⁵ and R⁶. R¹⁹ is a fluoroalkyl group. As the fluoroalkyl group, there may be mentioned a fluoroalkyl group exemplified in the said R¹ and R². Each of R¹⁴, R¹⁵ and R¹⁶ is the same as above. f denotes an integer of 1 to 6.

As an acryl monomer represented by the formula (11c), there may be mentioned, for example, a compound in which R¹⁴ is a trifluoromethyl group such as 1-nonafluorobutyl-4-(2-trifluoromethyl-2-propenoyloxy)cycl ohexane, 1-tridecafluorohexyl-4-(2-trifluoromethyl-2-propenoyloxy)cyclohexane, 1-nonafluorobutyl-4-(2-trifluoromethyl-2-propenoyloxy)norbornane, 2-tridecafluorohexyl-4-(2-trifluoromethyl-2-propenoyloxy)norbornane, 2-tridecafluorohexyl-5-(2-trifluoromethyl-2-propenoyloxy)-7-oxabicyclo[2.2.1]heptane, 2-tridecafluorohexyl-6-(2-trifluoromethyl-2-propenoyloxy)perhydronaphthalene, 3-tridecafluorohexyl-8-(2-trifluoromethyl-2-propenoyloxy)tetracyclo[4.4.0.1^{2,5}.1^{7,10}]dodecane, a compound corresponding to the said compound in which R¹⁴ is a fluorine atom, a hydrogen atom or a methyl group, and others.

In the formula (11d), a ring Z⁶ is an alicyclic carbon ring which may have a substituent. As the alicyclic carbon ring, there may be mentioned a ring represented the above formula (10a), (10b) or (10c) and so on. As a substituent which the alicyclic carbon ring may have there may be mentioned the same as a substituent which the said ring Z¹ may have. R²⁰ is a hydrogen atom, a fluorine atom, an alkyl group or a fluoroalkyl group. The said alkyl group and fluoroalkyl group are same as an alkyl group and fluoroalkyl group in R⁵ and R⁶. each of R¹⁴, R¹⁵ and R¹⁶ is the same as above. g denotes 3 or 4.

As an acryl monomer represented by the formula (11d), there may be mentioned, for example, a compound in which R¹⁴ is a trifluoromethyl group such as 1,1,2,2,3,3,3a,7a-octafluoro-5-(2-trifluoromethyl-2-propen oyloxy)perhydroindene, 2,3,3,4,4,5,5,6-octafluoro-8-(2-trifluoromethyl-2-propenoyloxy)tricyclo[5.2.1.0^{2,6}]decane, 2,3,3,4,4,5,5,6-octafluoro-8-(2-trifluoromethyl-2-propenoyloxy)-10-oxatricyclo[5.2.1.0^{2,6}]decane, 1,1,2,2,3,3,4,4,4a,8a-decafluoro-6-(2-trifluoromethyl-2-propenoyloxy)decalin, 2,3,3,4,4,5,5,6,6,7-decafluoro-9-(2-trifluoromethyl-2-propenoyloxy)tricyclo[6.2.1.0^{2,7}]undecane, a compound corresponding to the said compound in which R¹⁴ is a fluorine atom, a hydrogen atom or a methyl group, and others. In the said acryl monomer, (1) a compound in which a ring Z⁴ of a compound represented by the formula (11a) is a ring represented by the formula (10d), (10e), (10f), (10g), (10h) or (10i), (2) a compound in which a ring Z⁴ of a compound represented by the formula (11a) is a ring represented by the formula (10a), (10b) or (10c), and a ring Z⁴ is bonded by a group having the said polar group or a group having the polar group or W2 is a group having a polar group, (3) a compound in which R11 of a compound represented by the formula (11b) is a group having a polar group, (4) a compound in which a ring Z⁵ of a compound represented by the formula (11c) is bonded by a polar group or a compound having a polar group and (5) a compound in which a ring Z⁶ of a compound represented by the formula (11d) is bonded by a polar group or a compound having a polar group are subjected to co-polymerize as a co-mo0nomer to form a repeated unit having substrate adhesion and/or hydrophilicity function.

The said acryl monomer can be produced by a known method or by using a known reaction. The said corresponding acryl monomer can be obtained by, for example, allowing an unsaturated carboxylic acid having a fluorine atom or not having or a reactive derivative thereof (such as an acid halide, an acid anhydride and an ester) to react with a hydroxy compound by following a common esterification using a base, an acid catalyst or an ester exchanging catalyst to perform an esterification.

As another example of a polymerizable unsaturated monomer except for a monomer of the present invention used to give various functions as a resist to a polymeric compound of the present invention, there may be mentioned a cyclic unsaturated monomer represented by the formula (12a) or (12b). In such a cyclic unsaturated monomer, due to a compound of the formula (12a), etching resistance of a polymer can be improved, and due to a compound of the formula (12b), substrate adhesion can be improved. Further, in the said cyclic unsaturated monomer, when a ring has a fluorine atom or a compound having a fluorine atom, transparency against a light of wavelength 300 nm or less, particularly vacuum ultraviolet light can be improved and when a ring is bonded by the said polar group or a compound having a polar group, substrate adhesion and/or hydrophilicity function can be improved. In addition, when having an ester in which an oxygen atom constituting an ester is bonded by a tertiary carbon, acid-eliminating function can be improved. These cyclic unsaturated monomers can be used alone or by unifying two sorts or more.

In the formula, each of Y⁹ and Y¹⁰ is an alkyl group, an oxygen, a sulfur atom or non-bonding. Y¹¹ is an oxygen atom or a —NH— group. f denotes an integer of 0 to 3. An atom constituting a ring in the formulae (12a) and (12b) may have a substituent. The said alkylene group is the same as an alkylene group in Y⁴ and others. As a substituent which a ring may have, there may be mentioned the same as a substituent which a ring Z may have. When a number of substituent is two or more, these are allowed to unify together and thereby a ring of 4 or more members such as, for example, a cycloalkene ring and a lactone ring may be formed with a carbon atom constituting a ring. These rings may have a substituent such as a fluorine atom (the same substituent as a substituent which an atom constituting the said ring Z¹ may have).

As a typical example of a cyclic unsaturated monomer represented by the formula (12a), there may be mentioned, for example, norbornane (=bicyclo[2.2.1]-2-heptene), 5-carboxy-5-trifluoromethylbicyclo[2.2.1]-2-heptene, 5-t-butoxycarbonyl-5-trifluoromethylbicyclo[2.2.1]-2-heptene, 7-oxabicyclo[2.2.1]-2-heptene, tricyclo[4.3.0.1^{2,5}]-3-decene, tricyclo[4.4.0.1^{2,5}]-3-undecene, tetracyclo[4.4.0.1^{2,5}.^{17,10}]-3-dodecene, 4-oxatricyclo[5.2.1.0^{2,6}]-8-decene-3-on, 4-oxatricyclo[5.2.1.0^{2,6}]-8-decene-3,5-dion, and others.

As a typical example of a cyclic unsaturated monomer represented by the formula (12b), there may be mentioned, for example, maleic anhydride, 2-fluoro maleic anhydride, 2-trifluoromethyl maleic anhydride, maleimide, N-carboxymaleimide, N-methylmaleimide and so on. In the said cyclic unsaturated monomer, (1) a compound in which a ring of a compound represented by the formula (12a) is bonded by the said polar group or a compound having a polar group and (2) a compound represented by the formula (12b) are subjected to co-polymerize as a co-monomer to form a repeated unit having substrate adhesion and/or hydrophilicity function. The said cyclic unsaturated monomer can be produced by a known method or by using a known reaction.

In a polymeric compound of the present invention, a ratio of repeated unit corresponding to a compound represented by the formula (1a) or (1b), particularly isn't limited, is usually 1 to 99 mole % based on all monomer units constituting a polymer, preferably 5 to 95 mole %, more preferably 10 to 80 mole %, particularly about 20 to 70 mole %. A ratio of repeated unit having acid-eliminating function is, for example, 5 to 80 mole % based on all monomer units constituting a polymer, preferably about 10 to 60 mole %. A ratio of repeated unit having substrate adhesion and/or hydrophilicity function is, for example, 20 to 95 mole % based on all monomer units constituting a polymer, preferably about 40 to 90 mole %. Further, a ratio of a repeated unit corresponding to a vinyl ether monomer represented by the formula (9a) or (9b), a repeated unit corresponding to an acryl monomer represented by the formula (11a), (11b), (11c) or (11d) and a repeated unit corresponding to a cyclic unsaturated monomer represented by the formula (12a) or (12b) are can be properly selected according to a function had by each monomer unit. A total ratio of these repeated units is 1 to 99 mole % based on all monomer units constituting a polymer, preferably 5 to 95 mole %, more preferably 20 to 90 mole %, particularly about 30 to 80 mole %.

When a fluorine-atom-containing polymerizable unsaturated-monomer of the present invention in allowed to (co-)polymerize to obtain a polymerizable monomer, polymerization can be performed by solution polymerization, bulk polymerization, suspension polymerization, bulk-suspension polymerization and emulsion polymerization and other commonly used method used in the production of acryl polymer and others, and particularly solution polymerization is preferable. In the solution polymerization, dropwise polymerization may be applied in order to obtain homogeneous quality of polymer.

As a polymerization solvent, a conventional solvent can be used and there may be mentioned, for example, an ether (diethyl ether, glycol ethers such as propylene glycol monomethyl ether, and other linear-chain ethers, tetrahydrofuran, dioxane, and other cyclic ethers), an ester (methyl acetate, ethyl acetate, butyl acetate, ethyl lactate, a glycol ether esters such as propylene glycol monomethyl ether acetate and so on), a ketone (acetone, methyl ethyl ketone, methyl isobutyl ketone, cyclohexanone and so on), an amide (N,N-dimethylacetoamide, N,N-dimethylformamide and so on), a sulfoxide (dimethylsulfoxide and soon), an alcohol (methanol, ethanol, propanol and so on), a hydrocarbon (an aromatic hydrocarbon such as benzene, toluene and xylene, an aliphatic hydrocarbon such as hexane, an alicyclic hydrocarbon such as cyclohexane, and others), a mixed solvent thereof and so on. Further, as a polymerization initiator, a conventional polymerization initiator can be applied. A polymerization temperature can be appropriately selected, for example, in a scope of from about 30 to about 150° C.

A polymer obtained by polymerization can be purified by precipitation or reprecipitation. A solvent for precipitation or reprecipitation may be either an organic solvent or water and further a mixing solvent may be well. As an organic solvent used for precipitation or reprecipitation solvent, there may be mentioned, for example, a hydrocarbon (pentane, hexane, heptane, octane, and other aliphatic hydrocarbons; cyclohexane, methylcyclohexane, and other alicyclic hydrocarbons; benzene, toluene, xylene, and other aromatic hydrocarbons), a halogenated hydrocarbon (a halogenated aliphatic hydrocarbon such as methylene chloride, chloroform and carbon tetrachloride; a halogenated aromatic hydrocarbon such as chlorobenzene and dichlorobenzene, and so on), a nitro compound (such as nitromethane and nitroethane), a nitrile (such as acetonitrile and benzonitrile), an ether (a linear chain ether such as diethyl ether, diisopropyl ether and dimethoxyethane; a cyclic ether such as tetrahydrofuran and dioxane), a ketone (such as acetone, methyl ethyl ketone and diisobutyl ketone), an ester (such as ethyl acetate and butyl acetate), a carbonate (such as dimethyl carbonate, diethyl carbonate, ethylene carbonate and propylene carbonate), an alcohol (such as methanol, ethanol, propanol, isopropylalcohol and butanol), a carboxylic acid (such as acetic acid), a mixed solvent containing these solvent and so on.

A weight average molecular weight (Mw) of a polymeric compound is, for example, from about 1000 to about 500000, preferably from about 3000 to about 50000, and a molecular weight distribution (Mw/Mn) is, for example, from about 1.5 to about 2.5. Incidentally, the said Mn is a number average molecular weight and both Mn and Mw are in terms of polystyrene.

[Resin Compositions for Photoresist and Process for Producing a Semiconductor]

A photoresist resin composition of the present invention contains at least one polymeric compound of the said present invention and a photosensitive acid generator. Incidentally, the photoresist resin composition may contain a polymer except for the polymeric compound as far as adversary affected to the resist function.

As a photosensitive acid generator, a compound commonly used or conventional as effectively providing acid by the action of exposure, for example, a diazonium salt, an iodonium salt (for example, diphenyl iodo hexafluorophosphate and so on), a sulfonium salt (for example, triphenyl sulfonium hexafluoroantimonate, triphenyl sulfonium hexafluorophosphate, triphenyl sulfonium methane sulfonate and so on), a sulfonic acid ester [for example, 1-phenyl-1-(4-methylphenyl)sulfonyloxy-1-benzoylmethane, 1,2,3-trisulfonyloxymethylbenzene, 1,3-dinitro-2-(4-phenylsulfonyloxymethyl)benzene, 1-phenyl-1-(4-methylphenylsulfonyloxymethyl)-1-hydroxy-1-b enzoylmethane and soon], an oxathiazol derivative, s-triazine derivative, a disulfone derivative (such as diphenyldisulfone), an imide compound, an oxime sulfonate, diazonaphtoquinone, benzoin tosylate and others can be applied. These photosensitive acid generators can be used by alone or in combination of 2 or more sorts.

The amount of photosensitive acid generator can be appropriately selected depending on strength of the acid generated by photo-exposure, a ratio of each repeated unit of the polymer and others, and for example, from about 0.1 to about 30 part by weight, preferably from about 1 to about 25 part by weight, and preferably from about 2 to about 20 part by weight, relative to 100 part by weight of the polymeric compound.

A photoresist resin composition may contain, if necessary, an alkali soluble component such as an alkali soluble resin (for example, a novolac resin, a phenol resin, an imide resin, carboxyl group-containing resin and so on), a coloring agent (for example, dyes), an organic solvent (for example, such as hydrocarbons, halogenated hydrocarbons, alcohols, esters, amides, ketones, ethers, cellosolves, carbitols, glycol ether esters, and mixed solvent of them), a basic compound (such as a hindered amine), a detergent, a anti-soluble agent, a sensitizer, a stabilizer and others.

A photoresist resin composition obtained by these procedures is coated on a base or substrate, and dried, the applied film (resist film) is exposed to light (or, further baked after exposure) to form a latent pattern, and is subsequently developed to form a fine pattern with a high degree of precision.

As a base or substrate, there may be mentioned silicon wafer, metal, plastics, glass, ceramic and so on. The photoresist resin composition can be applied using a conventional application means such as a spin coater, a dip coater, a roller coater. The applied film has a thickness of, for example, from about 0.01 to about 20 μm, and preferably from about 0.05 to about 1 μm.

Light rays with different wavelengths such as ultraviolet rays and X-rays can be used in exposure. For example, g-light, i-light, excimer laser (for example, XeCl, KrF, KrCl, ArF, ArCl, F₂, Kr₂, KrAr, Ar₂ and so on) are usually used for semiconductor resist. An exposure energy is, for example, from about 0.1 to about 1000 mJ/cm².

Light irradiation allows the photosensitive acid generator to generate an acid, and the acid allows, for example, the eliminating portion of acid-eliminating group of the said polymeric compound to leave promptly and thereby yields a carboxyl group that contributes to solubilization. Therefore, development with water or an alkaline developing solution can yield a predetermined pattern with a high degree of precision.

### EXAMPLES

The present invention will be illustrated in more detail with reference to several examples below, which is not intended to limit the scope of the invention. Figures of the right-under brackets in the structural formulae of polymers denote % by mole of the prepared monomer corresponding to the repeated unit (monomer unit). A weight average molecular weight (Mw) and a molecular weight distribution (Mw/Mn) are measured by a GPC measurement using a refractometer (RI) and tetrahydrofuran (THF) as a detector and an eluent, respectively in terms of standard polystyrene. Three columns KF-806L (commercial name) manufactured by SHOUWA DENKO Inc. are connected in series to use for GPC and the reaction is performed under the condition comprising the column temperature of 40° C., RI temperature of 40° C., and eluent flow rate of 0.8 ml/min.

### Production Example 1

To a three-necked flask equipped with a thermometer, 11.6 g (0.1 mol) of 1,4-cyclohexandiol, 12.1 g (0.12 mol) of triethylamine and 200 ml of tetrahydrofuran was added and stirred under nitrogen stream while cool by ice. To this mixed liquid, 19.0 g (0.12 mol) of 2-trifluoromethyl acrylic acid chloride was added and stirred for 2 hours at room temperature. After the reaction, the reaction mixture was concentrated under reduced pressure and after 300 ml of pure water was added to the concentrated residue, extracted twice by 300 ml of ethyl acetate. The organic layers were united, washed respectively 300 ml of 5 weight % sodium bicarbonate aqueous solution and 300 ml of 10 weight % salt aqueous solution, dried by magnesium sulfate and concentrated under reduced pressure. By subjecting the concentrated residue to purify with a silicagel chromatography, 13.8 g (0.058 mol) of 2-trifluoromethyl acrylic acid 4-hydroxycyclohexyl [=1-(2-trifluoromethyl-2-propenoyloxy)-4-hydroxycyclohexane] represented by the following formula (17) can be obtained.

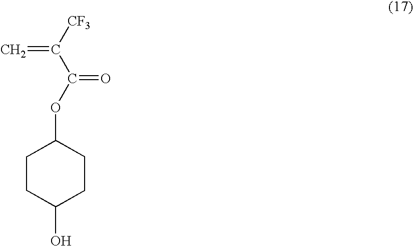

In addition, γ,γ-dimethyl-α-vinyloxy-γ-butyrolactone [the following formula (18)] used as a monomer in the following Examples was synthesized from α-hydroxy-γ,γ-dimethyl-γ-butyrolactone and vinyl acetate by following to the method described in Japanese Unexamined Patent Application Publication No. 2003-73321 and purified by a distillation under reduced pressure. 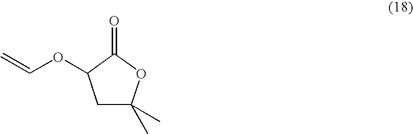
Further, 1-vinyloxy-4-oxatricyclo[4.3.1.1^{3,8}]undecane-5-on [the following formula (19)] was synthesized from 1-hydroxy-4-oxatricyclo[4.3.1.1^{3,8}]undecane-5-on and vinyl acetate by following to the method described in Japanese Unexamined Patent Application Publication No. 2003-73321 and purified by a distillation under reduced pressure, and was used.

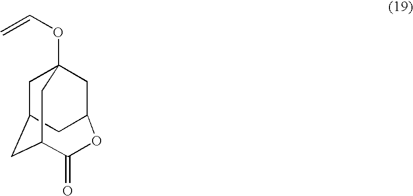

### Production Example 2

The reaction was performed under nitrogen atmosphere in all. To a three-necked flask equipped with a thermometer, 120 ml of 1M-3,3,4,4,5,5,6,6,6-nonafluorohexyl magnesium iodo ethylether solution was added and the mixed liquid of 15.0 g of 2-adamantanone and 200 ml of tetrahydrofuran was added by dropwise to this solution for one hour while stirred at room temperature. After dropping, the reaction mixture stirred for 4 hours while heating and refluxing. After the reaction, 200 ml of ethylether and 400 ml of saturated ammonium chloride aqueous solution were added to the reaction mixture, the organic layer was dried by magnesium sulfate and concentrated under reduced pressure. By allowing the concentrated residue to purification of a silicagel chromatography, 25 g of 2-(3,3,4,4,5,5,6,6,6-nonafluorohexyl)adamantane-2-ol [=2-(3,3,4,4,5,5,6,6,6-nonafluorohexyl)-2-hydrolxy]adamantane was obtained. In addition, 1M-3,3,4,4,5,5,6,6,6-nonafluorohexyl magnesium iodo ethylether solution as a raw material was synthesized from 1,1,1,2,2,3,3,4,4-nonafluoro-6-iodo hexane and magnesium by following to a conventional method (Grignard reaction) and was used.

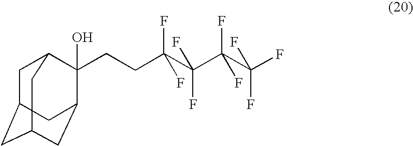

### Example 1

The reaction was performed under nitrogen atmosphere in all. To a three-necked flask equipped with a thermometer, 4.0 g of 2-(3,3,4,4,5,5,6,6,6-nonafluorohexyl)adamantane-2-ol, 3.0 g of triethylamine and 30 ml of tetrahydrofuran were added and to this solution, the mixed liquid of 4.0 g of 2-trifluoromethyl acrylic acid chloride and 20 ml of tetrahydrofuran was added by dropwise for 30 minutes while stirred at room temperature. After dropping, the reaction mixture was stirred for 6 hours at 50° C. After the reaction, 100 ml of 10 weight % salt aqueous solution was added and extracted twice by 100 ml of ethyl acetate. The organic layers were united, washed by 100 ml of saturated salt aqueous solution, dried by magnesium sulfate and concentrated under reduced pressure. By subjecting the concentrated residue to purify with a silicagel chromatography, 1.4 g of 2-trifluoromethyl acrylic acid 2-(3,3,4,4,5,5,6,6,6-nonafluorohexyl)adamantane-2-ylester 2-(3,3,4,4,5,5,6,6,6-nonafluorohexyl)-2-(2-trifluoromethyl-2-propenoyloxy)adamantane] represented by the following formula (21) can be obtained. 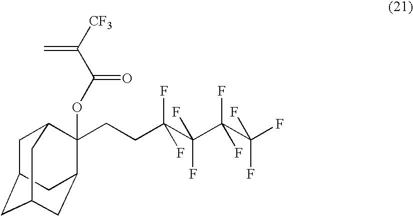
[Specter data of 2-trifluoromethyl acrylic acid 2-(3,3,4,4,5,5,6,6,6-nonafluorohexyl)adamantane-2-ylester]

¹H-NMR (CDCl³)) δ: 1.64 (m, 1H), 1.67 (m, 1H), 1.75-1.90 (m, 8H), 1.97-2.08 (m, 4H), 2.43 (m, 2H), 2.54 (quint, 2H), 6.37 (m, 1H), 6.71 (m, 1H)

### Example 2

The reaction was performed under nitrogen atmosphere in all. To a three-necked flask equipped with a thermometer, 24 ml of 1M-3,3,4,4,5,5,6,6,6-nonafluorohexyl magnesium iodo ethylether solution was added and the mixed liquid of 3 g of 2-adamantanone and 40 ml of tetrahydrofuran was added by dropwise to this solution for one hour while stirred at room temperature. After dropping, the reaction mixture stirred for 4 hours while heating and refluxing. The reaction mixture was stood and cooled until room temperature, and the mixed liquid of 3.2 g of 2-trifluoromethyl acrylic acid chloride and 10 ml of tetrahydrofuran was added by dropwise for one hour. After dropping, the reaction mixture was stirred for 5 hours while heating and refluxing. After the reaction, 200 ml of 10 weight % salt aqueous solution was added and extracted twice by 100 ml of ethyl acetate. The organic layers were united, washed by 100 ml of saturated salt aqueous solution, dried by magnesium sulfate and concentrated under reduced pressure. By subjecting the concentrated residue to purify with a silicagel chromatography, 2.2 g of 2-trifluoromethyl acrylic acid 2-(3,3,4,4,5,5,6,6,6-nonafluorohexyl)adamantane-2-ylester [=2-(3,3,4,4,5,5,6,6,6-nonafluorohexyl)-2-(2-trifluoromethyl-2-propenoyloxy) adamantane] represented by the above formula (21) can be obtained.

### Example 3

The reaction was performed under nitrogen atmosphere in all. To a three-necked flask equipped with a thermometer, 100 ml of 1M-3,3,4,4,5,5,6,6,6-nonafluorohexyl magnesium iodo ethylether solution was added. The mixed liquid of 14.6 g of 2-adamantanone and 300 ml of tetrahydrofuran was added by dropwise for 20 minutes while stirred at room temperature. After dropping, the reaction mixture stirred for 4 hours at 55° C. Then, after added 50 ml of p-methoxyphenol to the reaction mixture, the mixed liquid of 9.7 g of acrylic acid chloride and 10 ml of tetrahydrofuran was added by dropwise for 10 minutes. After dropping, the reaction mixture was stirred for 5 hours at 55° C. The reaction mixture was stood and cooled until room temperature, 30 ml of 10 weight % sodium carbonate aqueous solution was added and the mixed liquid of 3.2 g of 2-(trifluoromethyl)acrylic acid chloride and 10 ml of tetrahydrofuran was added by dropwise for one hour. After dropping, the reaction mixture was stirred for 5 hours while heating and refluxing. After the reaction, the reaction mixture was concentrated under reduced pressure, 400 ml of 10 weight % sodium carbonate aqueous solution was added to the concentrated residue and this was extracted three times by each of 400 ml of diisopropyl ether. The organic layers were united, washed by 200 ml of 10 weight % sodium carbonate aqueous solution and 200 ml of 10 weight % salt aqueous solution, dried by magnesium sulfate and concentrated under reduced pressure. By subjecting the concentrated residue to purify with a silicagel chromatography, 17.2 g of 2-(3,3,4,4,5,5,6,6,6-nonafluorohexyl)-2-(2-propenoyloxy)ad amantane represented by the following formula (22) can be obtained. 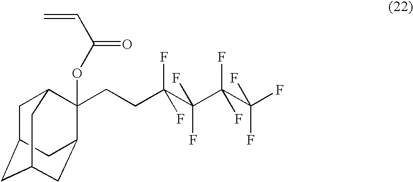
[Specter data of 2-(3,3,4,4,5,5,6,6,6-nonafluorohexyl)-2-(2-propenoyloxy)ad amantane]

¹H-NMR (CDCl³)) δ: 1.61 (m, 1H), 1.64 (m, 1H), 1.74-1.80 (m, 4H), 1.84-1.89 (m, 4H), 1.96-2.07 (m, 4H), 2.41 (m, 2H), 2.50-2.54 (quint, 2H), 5.80-5.82 (d, 1H), 6.09-6.13 (dd, 1H), 6.35-6.39 (d, 1H)

### Example 4

Production of polymeric compound of the following structure

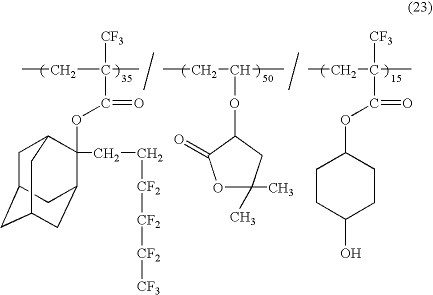

In a 100 ml round-bottom flask equipped with a reflux condenser, a stirrer and three-way cock, 6.21 g (11.9 mmol) of 2-(3,3,4,4,5,5,6,6,6-nonafluorohexyl)-2-(2-trifluoromethyl-2-propenoyloxy)adamantane, 2.66 g (17.0 mmol) of γ,γ-dimethyl-α-vinyloxy-γ-butyrolactone, 1.14 g (5.1 mmol) of 1-(2-trifluoromethyl-2-propenoyloxy)-4-hydroxycyclohexane and 0.10 g of initiator [manufactured by WAKO JUNYAKU INDUSTRY Inc., trade name “V-65”] were placed, and was dissolved in 5.0 g of propylene glycol monomethyl ether acetate (PGMEA). Subsequently, after replacing with dry nitrogen gas in the flask, and was stirred for 3 hours in an atmosphere of nitrogen gas while maintaining the temperature of a reaction system at 60° C. The reaction solution was diluted with 30.0 g of tetrahydrofuran, and the solution was dropped into 500 g of a mixed solution of 450 g of hexane and 50 g of ethyl acetate, the produced precipitate was purified by filtration. The recovered precipitate was dried under reduced pressure, was dissolved in 35 g of tetrahydrofuran, and subsequently was dropped into 500 g of a mixed solution of 450 g of hexane and 50 g of ethyl acetate, and the obtained precipitate was removed, and the purification was repeated according to the above procedure. The amount of the polymer obtained after drying under reduced pressure was 8.2 g. The polymer was analyzed by GPC analysis and was found to have a weight average molecular weight of 6800 in terms of the standard polystyrene and a molecular weight distribution of 1.95. In addition, in the result of ¹³C-NMR (in CDCl₃) analysis, the composition of polymer was in the ratio of in the ratio of 34:45:21 (ratio by mole) (in order of from left of the structural formula).

### Example 5

Production of polymeric compound of the following structure

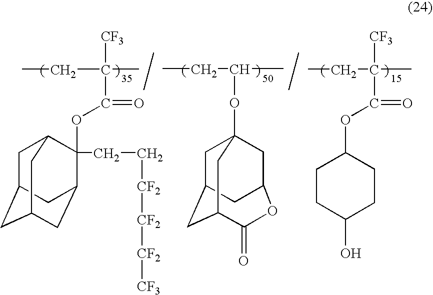

The polymeric compound was synthesized according to the same procedure as Example 3 except for using 5.70 g (11.0 mmol) of 2-(3,3,4,4,5,5,6,6,6-nonafluorohexyl)-2-(2-trifluoromethyl-2-propenoyloxy)adamantane, 3.26 g (15.7 mmol) of 1-vinyloxy-4-oxatricyclo[4.3.1.1^{3,8}]undecane-5-on, and 1.04 g (4.7 mmol) of 1-(2-trifluoromethyl-2-propenoyloxy)-4-hydroxycyclohexane as monomers of raw material. The amount of the polymer obtained after drying the recovered precipitate under reduced pressure was 6.3 g. The polymer was analyzed by GPC analysis and was found to a weight average molecular weight of 7200 in terms of standard polystyrene and a molecular weight distribution of 2.05. In addition, in the result of ¹³C-NMR (in CDCl₃) analysis, the composition of polymer was in the ratio of in the ratio of 33:49:18 (ratio by mole) (in order of from left of the structural formula

### Example 6

Production of polymeric compound of the following structure

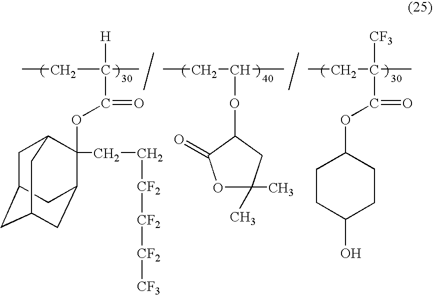

In a 100 ml round-bottom flask equipped with a reflux condenser, a stirrer and three-way cock, 5.03 g (11.1 mmol) of 2-(3,3,4,4,5,5,6,6,6-nonafluorohexyl)-2-(2-propenoyloxy)ad amantane, 2.32 g (14.8 mmol) of γ,γ-dimethyl-α-vinyloxy-γ-butyrolactone, 2.65 g (11.1 mmol) of 1-(2-trifluoromethyl-2-propenoyloxy)-4-hydroxycyclohexane and 0.10 g of initiator [manufactured by WAKO JUNYAKU INDUSTRY Inc., trade name “V-65”] were placed, and was dissolved in 5.0 g of tetrahydrofuran. Subsequently, after replacing with dry nitrogen gas in the flask, and was stirred for 3 hours in an atmosphere of nitrogen gas while maintaining the temperature of a reaction system at 60° C. The reaction solution was diluted with 30.0 g of tetrahydrofuran, and the solution was dropped into 500 g of a mixed solution of 450 g of hexane and 50 g of ethyl acetate, the produced precipitate was purified by filtration. The recovered precipitate was dried under reduced pressure, was dissolved in 35 g of tetrahydrofuran, and subsequently was dropped into 500 g of a mixed solution of 450 g of hexane and 50 g of ethyl acetate, and the obtained precipitate was removed, and the purification was repeated according to the above procedure. The amount of the polymer obtained after drying under reduced pressure was 6.5 g. The polymer was analyzed by GPC analysis and was found to have a weight average molecular weight of 7400 in terms of the standard polystyrene and a molecular weight distribution of 1.95. In addition, in the result of ¹³C-NMR (in CDCl₃) analysis, the composition of polymer was in the ratio of in the ratio of 25:42:33 (ratio by mole) (in order of from left of the structural formula).

### Example 7

Production of polymeric compound of the following structure

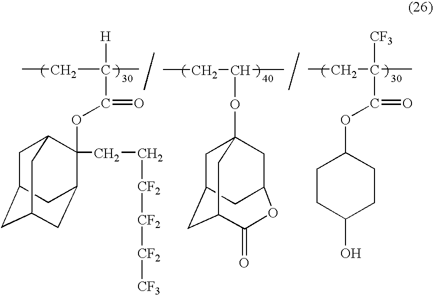

In a 100 ml round-bottom flask equipped with a reflux condenser, a stirrer and three-way cock, 4.67 g (10.3 mmol) of 2-(3,3,4,4,5,5,6,6,6-nonafluorohexyl)-2-(2-propenoyloxy)ad amantane, 2.86 g (13.8 mmol) of 1-vinyloxy-4-oxatricyclo[4.3.1.1^{3,8}]undecane-5-on, 2.46 g (10.3 mmol) of 1-(2-trifluoromethyl-2-propenoyloxy)-4-hydroxycyclohexane and 0.10 g of initiator [manufactured by WAKO JUNYAKU INDUSTRY Inc., trade name “V-65”] were placed, and was dissolved in 5.0 g of tetrahydrofuran. Subsequently, after replacing with dry nitrogen gas in the flask, and was stirred for 3 hours in an atmosphere of nitrogen gas while maintaining the temperature of a reaction system at 60° C. The reaction solution was diluted with 30.0 g of tetrahydrofuran, and the solution was dropped into 500 g of a mixed solution of 450 g of hexane and 50 g of ethyl acetate, the produced precipitate was purified by filtration. The recovered precipitate was dried under reduced pressure, was dissolved in 35 g of tetrahydrofuran, and subsequently was dropped into 500 g of a mixed solution of 450 g of hexane and 50 g of ethyl acetate, and the obtained precipitate was removed, and the purification was repeated according to the above procedure. The amount of the polymer obtained after drying under reduced pressure was 6.2 g. The polymer was analyzed by GPC analysis and was found to have a weight average molecular weight of 6800 in terms of the standard polystyrene and a molecular weight distribution of 1.92. In addition, in the result of ¹³C-NMR (in CDCl₃) analysis, the composition of polymer was in the ratio of in the ratio of 23:41:36 (ratio by mole) (in order of from left of the structural formula).

Valuation Test

(Transparency of Polymer)

1 g of each of the polymers obtained in the above Examples 4 to 7 was dissolved in 10 g of propylene glycol monomethyl ether acetate (PGMEA), and was filtered through a filter of 0.2 μm to prepare a polymer solution. The polymer solution was applied onto a MgF₂ substrate by spin coating, was baked on a hot plate at a temperature of 100° C. for 120 seconds to form a polymer film 100 nm thick. Light transparency at 157 nm wavelength of the film was measured by using a vacuum-ultraviolet photometer [manufactured by NIHON BUNKO Inc., VUV-200S] and was found to be 45% or more in any case.

(Preparation of Resist and Formation of Pattern)

100 parts by weight of each of the polymers obtained in the above Example 4 to 7 and 10 parts by weight of triphenylsulfonium hexafluoroantimonate were mixed with a solvent propyleneglycol monomethyl ether acetate (PGMEA) to prepare a photoresist resin composition of 17% by weight polymer-concentration. This composition was applied onto a silicon wafer by spin coating method to form a photosensitive layer of 1.0-μm thickness. The photosensitive layer was subjected to prebaking on a hot plate at a temperature of 100° C. for 150 seconds and was exposed to light through a mask using KrF excimer laser having a wavelength of 247 nm at an irradiance of 30 mJ/cm². The exposed layer was then subjected to post-exposure baking at a temperature of 100° C. for 60 seconds; was subjected to development in a 0.3 M aqueous tetramethylammonium hydroxide solution for 60 seconds; and was rinsed with pure water to yield a pattern with a 0.20-μm line and space in any case.

1. A fluorine-atom-containing polymerizable unsaturated-monomer represented by the following formula (1a) or (1b): 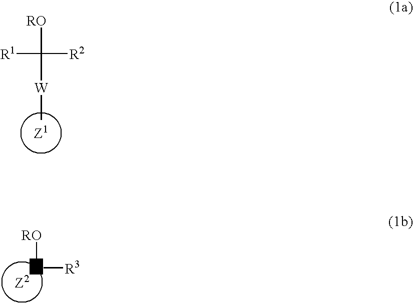
wherein each of a ring Z¹ and a ring Z² is an alicyclic carbon ring; when the alicyclic carbon ring is multi-cyclic, a part of atoms constituting the ring may be substituted by an oxygen atom, a sulfur atom or a nitrogen atom; an atom constituting the ring Z¹ or the ring Z² may have a substituent; each of R¹, R² and R³ is an alkyl group or a fluoroalkyl group; W denotes a single bond or a combining group and R is an unsaturated acyl group represented by the following formula (2): 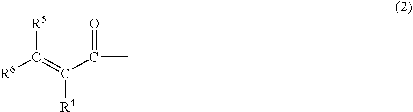
wherein each of R⁴, R⁵ and R⁶ is a hydrogen atom, a fluorine atom, an alkyl group or a fluoroalkyl group; a carbon atom bonded by at least one hydrogen atom exists in an adjacent position to the carbon atom bonded by an RO— group represented by the formula (1a) and the formula (1b); provided that at least one of R¹, R², R⁴, R⁵ and R⁶ is a fluorine atom or a fluoroalkyl group in the formula (1a), and in the formula (1b) (i) the ring Z² is bonded by a fluorine atom or a fluoroalkyl group, or (ii) R³ is a fluoroalkyl group.

2. A fluorine-atom-containing polymerizable unsaturated-monomer according to claim 1, wherein neither of adjacent carbon atoms to the carbon atom bonded by an RO— group has a fluorine atom in the formula (1a) or the formula (1b).

3. A fluorine-atom-containing polymerizable unsaturated-monomer according to claim 1 or claim 2, wherein, in the formula (1a),
(i) the ring Z¹ is bonded by a fluorine atom or a fluoroalkyl group;
(ii) at least one of R¹ and R² is a fluoroalkyl group having a hydrogen atom at 1 st position;
(iii) both of R¹ and R² are alkyl groups which have a hydrogen atom at 1 st position and may be fluorinated; or
(iv) at least one of R¹ and R² is an alkyl group of which a carbon number is three or more and may be fluorinated.

4. A fluorine-atom-containing polymerizable unsaturated-monomer according to claim 1, wherein the ring Z¹ or the ring Z² is a ring represented by the following formula (3a), (3b) or (3c); 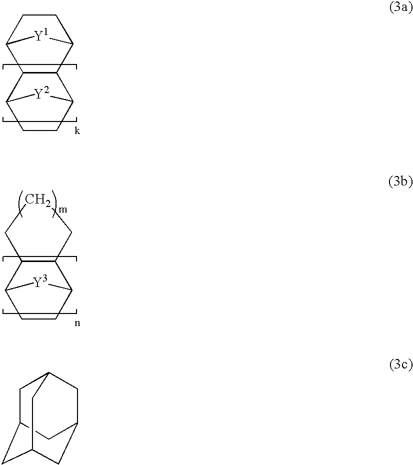
wherein Y¹ is an alkylene group, an oxygen atom or a sulfur atom, each of Y² and Y³ is an alkylene group, an oxygen atom, a sulfur atom or non bonding, each of k and n denotes an integer of 0 to 3, m denotes 1 or 2, and an atom constituting the rings in the formulae may have a substituent.

5. A process for producing a fluorine-atom-containing polymerizable unsaturated-monomer wherein a compound represented by the following formula (1a): 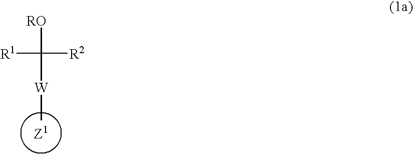
wherein each of the ring Z¹ is an alicyclic carbon ring, R¹ is an alkyl group or a fluoroalkyl group, R² is an alkyl group or a fluoroalkyl group, R is an unsaturated acyl group represented by the following formula (2): 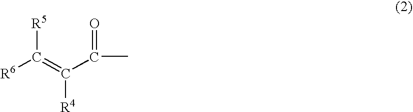
wherein each of R⁴, R⁵ and R⁶ is a hydrogen atom, a fluorine atom, an alkyl group or a fluoroalkyl group; and W is the same as above; provided that, when a compound represented by the formula (4b) is used as a raw material, R¹═R²; and a carbon atom bonded by at least one hydrogen atom exists in an adjacent position to the carbon atom bonded by an RO— group;
is obtained by allowing a compound represented by the following formula (4a) or (4b): 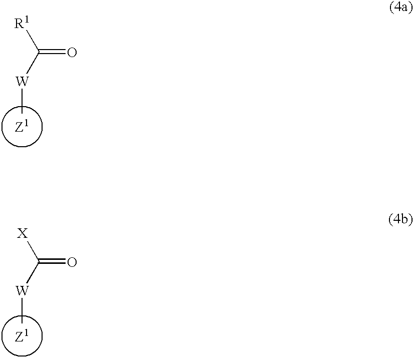
wherein a ring Z¹ is the same as above; when the alicyclic carbon ring is multi-cyclic, a part of atoms constituting the ring may be substituted by an oxygen atom, a sulfur atom or a nitrogen atom; an atom constituting the ring Z¹ may have a substituent; R¹ is the same as above; X is a halogen atom; W denotes a single bond or a combining group;
to react with a (fluoro) alkylating agent represented by the following formula (5):
R²-M (5)
wherein R is the same as above; M is a metal atom or a —MgX¹ group, wherein X¹ is a halogen atom; provided that a carbon atom bonded by at least one hydrogen atom exists in a adjacent position to a carbonyl group in the above formula (4a) or (4b), or in 1st position of R² in the formula (5);
and allowing an acyl halide represented by the following formula (6) to react to the obtained reaction product:
R—X² (6)
wherein R is the same as above; and X² is a halogen atom; provided that at least one of R¹, R², R⁴, R⁵ and R⁶ is a fluorine atom or a fluoroalkyl group.

6. A process for producing a fluorine-atom-containing polymerizable unsaturated-monomer wherein a compound represented by the following formula (1b): 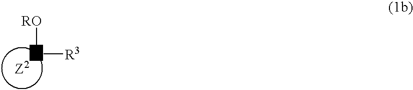
wherein the ring Z² is an alicyclic carbon ring; when the alicyclic carbon ring is multi-cyclic, a part of atoms constituting the ring may be substituted by an oxygen atom, a sulfur atom or a nitrogen atom; an atom constituting the ring Z² may have a substituent, R is an alkyl group or a fluoroalkyl group, and R is an unsaturated acyl group represented by the following formula (2): 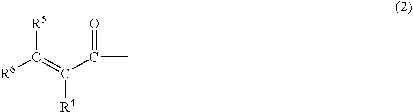
wherein each of R⁴, R⁵ and R⁶ is a hydrogen atom, a fluorine atom, an alkyl group or a fluoroalkyl group; and a carbon atom bonded by at least one hydrogen atom exists in an adjacent position to the carbon atom bonded by an RO— group;
is obtained by allowing a carbonyl compound represented by the following formula (7): 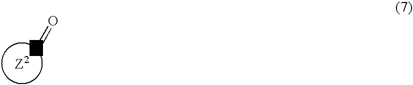
wherein a ring Z² is the same as above, to react with a (fluoro) alkylating agent represented by the following formula (8):
R²-M (8)
wherein R³ is the same as above; M is a metal atom or a —MgX¹ group, wherein X¹ is a halogen atom; provided that a carbon atom bonded by at least one hydrogen atom exists in a adjacent position to a carbonyl group in the above formula (7) or in 1st position of R³ in the formula (8); and allowing an acyl halide represented by the following formula (6) to react to the obtained reaction product:
R_{—X}² (6)
wherein R is the same as above; and X² is a halogen atom.

7. A polymeric compound having a repeated unit corresponding to a fluorine-atom-containing polymerizable unsaturated-monomer described in claim 1.

8. A polymeric compound according to claim 7, wherein a repeated unit having a substrate-adhesive function and/or a hydrophilic function is contained.

9. A polymeric compound according to claim 7, wherein a repeated unit corresponding to a vinyl ether monomer represented by the following formula (9a) or (9b): 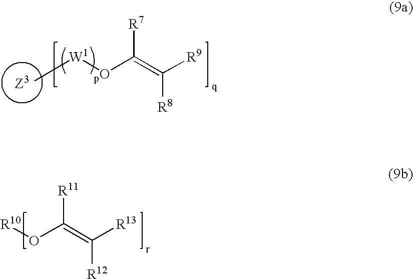
wherein, in the formula (9a), the ring Z³ is one of rings represented by the following formula (10a), (10b), (10c), (10d), (10e), (10f), (10g), (10h) or (10i): 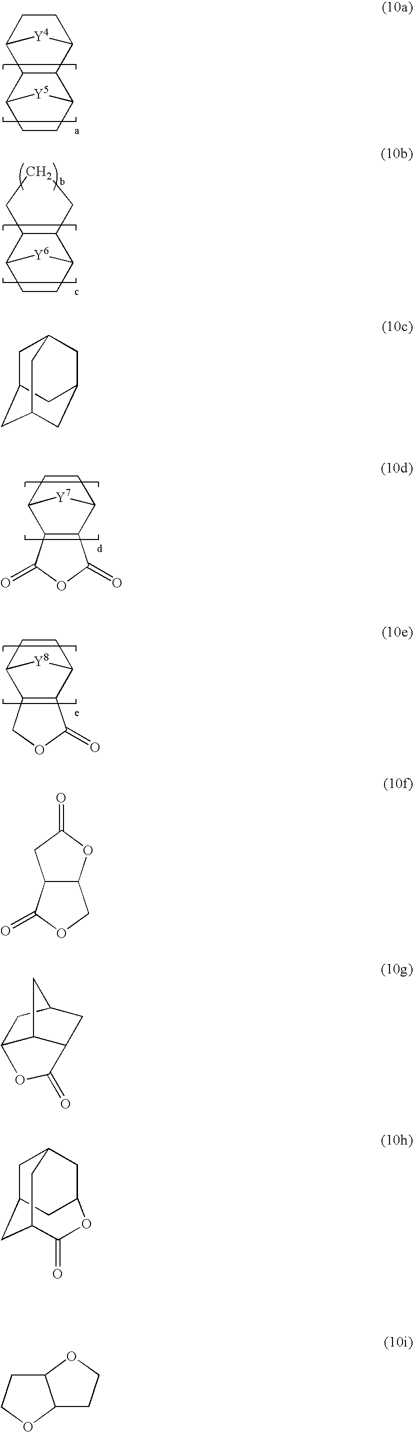
wherein Y⁴ is an alkylene group, an oxygen atom or a sulfur atom; each of Y⁵, Y⁶, Y⁷ and Y⁸ is an alkylene group, an oxygen atom, sulfur atom or non-bonding; each of a, c, d and e denotes an integer of 0 to 3; b denotes 1 or 2; and rings of the formulae may have a substituent; W¹ is a bivalent hydrocarbon group; each of R⁷, R⁸ and R⁹ is identical to or different from a hydrogen atom or an organic group; at least two of a ring Z³, W¹, R⁷, R⁸ and R⁹ may be combined together to constitute a ring with one or two or more adjacent atoms; p denotes 0 or 1 and q denoted an integer 1 to 8; when q is 2 or more, each of groups in q pairs of brackets may be identical to or different from; in the formula (9b), R¹⁰ is an alkyl group which may have a substituent and each of R¹¹, R¹² and R¹³ is identical to or different from a hydrogen atom or an organic group; at least two of R¹⁰, R¹¹, R¹² and R¹³ may be combined together to constitute a ring with one or two or more adjacent atoms; r denotes an integer of 1 to 8; when r is 2 or more, each of groups in r pairs of brackets may be identical to or different from;
is contained.

10. A polymeric compound according to claim 7 or 9, wherein a repeated unit corresponding to an acryl monomer represented by the following formula (11a), (11b), (11c) or (11d): 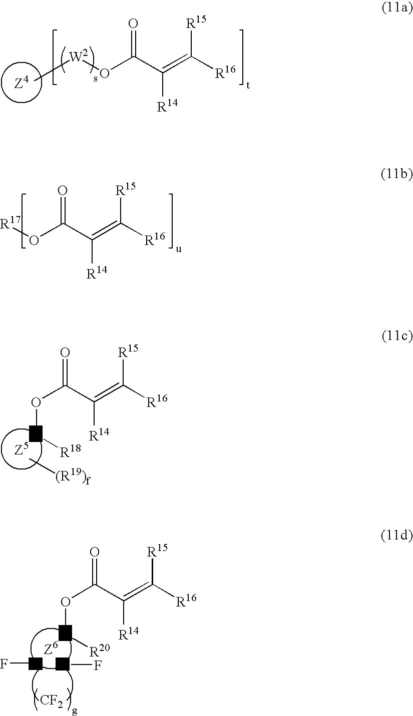
wherein in the formula (11a), the ring Z⁴ is one of rings represented by the following formula (10a), (10b), (10c), (10d), (10e), (10f), (10g), (10h) or (10i): 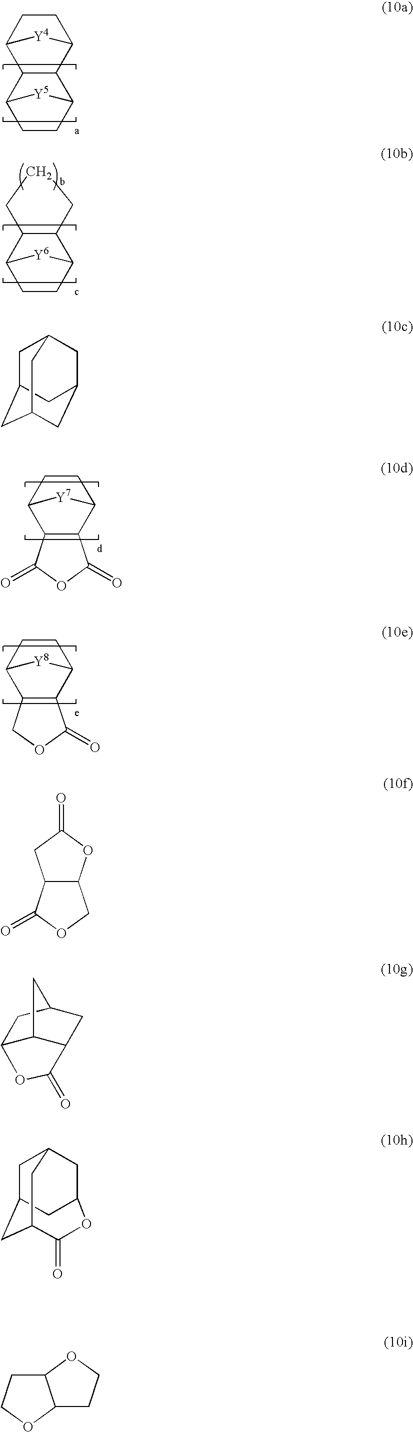
wherein Y⁴ is an alkylene group, an oxygen atom or a sulfur atom; each of Y⁵, Y⁶, Y⁷ and Y⁸ is an alkylene group, an oxygen atom, sulfur atom or non-bonding; each of a, c, d and e denotes an integer of 0 to 3; b denotes 1 or 2; and rings of the formulae may have a substituent; W is a bivalent hydrocarbon group; provided that each of Z⁴ and W² does not have a fluorine atom or a fluoroalkyl group as a substituent; each of R¹⁴, R¹⁵ and R¹⁶ is a hydrogen atom, a fluorine atom, an alkyl group or a fluoroalkyl group; s denotes 0 or 1 and t denotes an integer of 1 to 8; when t is 2 or more, each of groups in t pairs of brackets may be identical to or different from; in the formula (11b), R¹⁷ is an alkyl group which may have a substituent and each of R¹⁴, R¹⁵ and R¹⁶ is the same as above; u denotes an integer of 1 to 8; when u is 2 or more, each of groups in u pairs of brackets may be identical to or different from; in the formula (11c), a ring Z⁵ is an alicyclic carbon ring which may have a substituent; R¹⁸ is a hydrogen atom, a fluorine atom, an alkyl group or a fluoroalkyl group and R¹⁹ is a fluoroalkyl group; each of R¹⁴, R¹⁵ and R¹⁶ is the same as above; f denotes an integer of 1 to 6; in the formula (11d), a ring Z⁶ is an alicyclic carbon ring which may have a substituent; R²⁰ is a hydrogen atom, a fluorine atom, an alkyl group or a fluoroalkyl group; each of R¹⁴, R¹⁵ and R¹⁶ is the same as above; g denotes 3 or 4; provided that compounds represented by the formulae (I a) and (lb) are excepted for;
is contained.

11. A polymeric compound according to claim 7, wherein a repeated unit corresponding to a cyclic unsaturated monomer represented by the following formula (12a) or (12b); 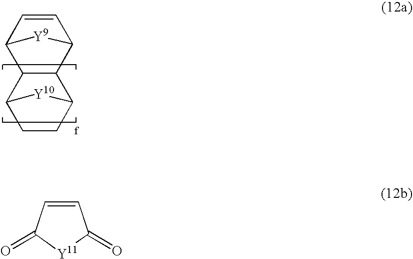
wherein each of Y9 and Y10 is an alkylene group, an oxygen atom, a sulfur atom or a non-bonding; Y11 is an oxygen atom or a —NH— group; f denotes an integer of 0 to 3; an atom constituting a ring of the formulae may have a substituent.

12. A photoresist resin composition, comprising at least the polymeric compound as claimed in claim 7 and a photo acid generator.

13. A process of producing a semiconductor, comprising the steps of applying the photoresist resin composition as claimed in claim 12 onto a base or substrate to form a resist film, exposing, developing and thereby produce a pattern.
